(19) United States
(12) Patent Application Publication
(10) Pub. No.: US 2023/0063821 A1
(43) Pub. Date: Mar. 2, 2023

(54) THROMBECTOMY CAPTURE SYSTEM

(71) Applicants: Karl V. Ganske, Hopkins, MN (US); Steve Michael, Eden Prairie, MN (US); Jacob Chmielewski, Clear Lake, MN (US); Alex Marine, Eden Prairie, MN (US)

(72) Inventors: Karl V. Ganske, Hopkins, MN (US); Steve Michael, Eden Prairie, MN (US); Jacob Chmielewski, Clear Lake, MN (US); Alex Marine, Eden Prairie, MN (US)

(21) Appl. No.: 17/896,589

(22) Filed: Aug. 26, 2022

Related U.S. Application Data
(60) Provisional application No. 63/237,958, filed on Aug. 27, 2021.
(60) Provisional application No. 63/348,128, filed on Jun. 2, 2022.

(51) Int. Cl. A61B 17/221

(52) U.S. Cl. CPC A61B 17/221; A61B 2017/2212; A61B 2017/22038

(57) ABSTRACT

An over-the-wire retrieval system having at least one expandable filter basket deployable and retrievable over a positioning guidewire that remains in place throughout the thrombectomy procedure. The filter catheter can slidably receive the positioning guidewire allowing the positioning guidewire to navigate a collapsed filter basket past a clot before the filter basket is expanded and pulled through the clot to engage or capture clot material. The filter basket can be re-collapsed and navigated past the clot over the positioning guidewire for multiple passes through the clot. The filter catheter can be paired with a flexible capture sleeve or an aspiration sheath for collection and removal of the clot material gathered by the filter basket on each pass. The flexible capture sleeve includes, in an example, a thrombus conveyor configured to transition between everted and inverted configurations.

### PRIORITY APPLICATIONS

This application claims the benefit of priority to U.S. Provisional Patent Application Ser. No. 63/237,958, filed Aug. 27, 2021 and U.S. Provisional Patent Application Ser. No. 63/348,128, filed Jun. 2, 2022, the contents of both which are incorporated herein by reference in their entireties.

### CROSS-REFERENCE TO RELATED PATENT DOCUMENTS

This patent application is also related to U.S. application Ser. No. 12/738,702, entitled INTRAVASCULAR GUIDEWIRE FILTER SYSTEM FOR PULMONARY EMBOLISM PROTECTION AND EMBOLISM REMOVAL OR MACERATION filed on Apr. 19, 2010, is herein incorporated by reference.

### TECHNICAL FIELD

This document pertains generally, but not by way of limitation, to an intravascular system, and more particularly, is for an intravascular filter system for clot protection, removal, or maceration.

### BACKGROUND

Thrombectomy is a surgical procedure used to remove a blood clot from a vessel, such as an artery or vein. A thrombectomy procedure can target a blood clot such as a thrombus, which is a clot formed in situ within the blood vessel. Alternatively, thrombectomy procedures can also target blood clots such as embolisms, which are thrombus that have dislodged from the original formation site and has become relodged in a different region of the vascular system. A pulmonary embolism (“PE”) is a specific type of embolism that is dislodged from a formation site, often the lower leg, and has relodged in a blood vessel within the lungs.

Mechanical thrombectomy procedures can involve deployment of a capture device at the clot. The capture device is then pulled through the clot to mechanically capture or entrain the clot for removal through a separate retrieval catheter or sheath. If the clot material is too organized or too large, the capture device may be unable to fully capture or entrain the clot material with a single pass of the capture device and may require multiple passes of the capture device through the clot to fully remove the clot or sufficiently clear the blood vessel to restore blood flow. While multiple passes of the capture device can remove organized and large clot, each deployment of the capture device typically requires multiple deployment steps to position the capture device and pass the capture device through the clot. For example, the capture device must be navigated past the clot in a collapsed configuration before being expanded and pulled through the clot to complete a pass. The multiple deployment steps required between each pass can substantially increase the overall length of each surgical procedure, cause injury to the patient, and increase the likelihood of complications.

Aspiration thrombectomy systems can involve placing a catheter or sheath adjacent the clot and drawing a vacuum through the catheter to pull the clot material into the catheter and out of the body. While aspiration thrombectomy systems are less affected by large or organized clots, aspiration thrombectomy systems often remove large amounts of blood from the patient to effectively remove the clot material. The removed blood must be replaced by substituting the removed blood or by using a separate system filter the removed blood and recirculate the filtered blood to the patient. In either case, the large quantity of removed blood likewise creates a risk of injury and complication for the patient.

### SUMMARY

The present inventors have recognized, among other things, that a problem to be solved can include efficiently making multiple passes through a clot with a mechanical clot capture device. In an example, the present subject matter can provide a solution to this problem, such as by an “over-the-wire” retrieval system having at least one expandable filter basket deployable and retrievable over a positioning guidewire that remains in place throughout the thrombectomy procedure. The retrieval system can comprise a filter assembly with an expandable filter basket mounted on a filter catheter defining a central lumen for slidably receiving the positioning guidewire. The positioning guidewire is a guide for rapidly navigating the filter basket past the clot in a collapsed configuration before the filter basket is expanded and pulled through the clot. The positioning guidewire can remain in place and in the same position for multiple passes through the clot, which improves the overall procedure time and reduces the risk of complication. The filter catheter can be paired with a flexible capture sleeve or an aspiration sheath for collection and removal of the clot material gathered by the filter basket on each pass.

A retrieval system, according to an embodiment, can comprise a flexible capture sleeve positioned at the distal end of a flexible sleeve positioning tube and also comprises a filter assembly mounted at the distal end of a filter catheter. The filter catheter defines a central lumen for slidably receiving a positioning guidewire for delivery and retrieval of the filter catheter over the positioning guidewire. Likewise, the flexible sleeve positioning tube defines a lumen for slidably receiving the filter catheter for delivery and retrieval over the filter catheter. In this configuration, the components of the retrieval system can be delivered, treat the clot, and retrieved over the positioning guidewire in a telescoping fashion while the positioning guidewire remains in place during each pass of through the clot. This permits efficient deployment and redeployment of the filter assembly distal to the clot for repeat passes through the clot without removing or repositioning of the positioning guidewire for each pass.

A retrieval system, according to an embodiment, can comprise a filter assembly mounted on a filter catheter and an aspiration sheath. The filter assembly can be expanded within a blood vessel distal to the clot and pulled through the clot to pull clot material proximate to the mouth of the aspiration sheath. A vacuum can be drawn to pull the collect clot material into the aspiration sheath. The filter catheter defines a central lumen for slidably receiving a positioning guidewire for delivery and retrieval of the filter catheter over the positioning guidewire. A filter delivery catheter can be slid over the filter catheter to collapse the filter assembly for navigating the filter assembly past the clot for redeployment distal to the clot. In this configuration, the filter assembly can be pulled through the clot for multiple rapid passes through the clot without fully removing the filter delivery catheter from the body or repositioning of the guidewire.

The present inventors have recognized, among other things, that a problem to be solved can include minimizing compression of thrombus that may initiate plugging in catheters during thrombus removal while enhancing removal of thrombus from vessels. Thrombus removal catheters are navigated to treatment sites and include profiles generally smaller than the vessels to facilitate navigation. In example catheters including mechanical extraction features, like baskets, the basket is delivered from the catheter and deployed, for instance distally relative to the thrombus. The basket is retracted toward the catheter and pulls the thrombus into the catheter lumen at the catheter mouth. In one example, the thrombus is aspirated into the catheter with a negative pressure. In another example, the thrombus is pulled into the catheter mouth through retraction of the basket. In each example, the thrombus is translated toward the catheter mouth and optionally compressed toward the catheter lumen proximate to the catheter mouth. In some instances, pulling the thrombus toward the catheter mouth causes the thrombus to compress against the mouth of the catheter forming a dense plug that clogs the catheter lumen, potentially snags the basket with the plugged thrombus (described herein), and frustrates removal of the thrombus. A clinician may in some examples forcefully retract the basket to attempt to further compress the plug and drive the plug into the catheter lumen. In some examples, the forceful retraction may cause damage to the basket or the thrombus catheter, and potentially shear some of the thrombus away from the thrombus catheter and into the vessel. In other examples, the thrombus catheter is removed with a surgical thrombectomy procedure (also referred to as a venous cut-down) including cutting of the vessel to facilitate removal of the catheter.

In other examples, a sleeve is provided as an intermediate feature to facilitate capture of thrombus without plugging. The basket is deployed from a delivery catheter, for instance distal to a thrombus. The sleeve is deployed from the delivery catheter and expands relative to the catheter lumen. The basket is moved proximally and pulls the intervening thrombus into the sleeve. The sleeve, thrombus and basket are in some examples withdrawn into the catheter lumen. Withdrawal toward the catheter mouth compresses the sleeve proximate to the catheter mouth and compresses the thrombus therein. In some examples, the compressed thrombus forms a dense plug in the sleeve and still frustrates further withdrawal into the catheter lumen.

Further, even before withdrawal of the catheter, basket and thrombus from the patient the relative movement of the thrombus along and within the sleeve in some examples initiates snagging between protein strands (fibrin) of the thrombus and the sleeve. The snagged thrombus in combination with proximal movement of the thrombus into the sleeve initiates and promotes compression of the sleeve around the thrombus because of the relative movement of the snagged thrombus relative to the sleeve. The compressed sleeve further compounds compression of the thrombus and in some examples further aggravates the development of dense thrombus plugs that frustrate withdrawal of the sleeve and thrombus into a catheter (e.g., for extraction from the patient).

In other examples, an everted tractor of woven, knitted or braided fibers extends from an inner catheter and is turned inside out (everted) and rolled along an exterior of a distal end of a delivery catheter. The distal end of the delivery catheter is positioned proximate to a thrombus, and the inner catheter connected with the tractor is moved proximally to unroll or invert the tractor while it contacts the thrombus. The fibers of the everted tractor move to the interior of the delivery catheter while unrolling (inverting) and mechanically engage and translate along the thrombus and pull the thrombus into the delivery catheter. The everted tractor relies on cohesiveness of the thrombus (and in some examples snagging of thrombus fibrin) to pull in distal portions of the thrombus as the tractor is unrolled. In some examples with less cohesive thrombus, the tractor may fail to pull in the distal portions of the thrombus if thrombus separates during the procedure. After inversion of the tractor the delivery catheter, inner catheter, and the tractor and thrombus are removed from the patient. The clinician may then reset the tractor along the delivery catheter through careful positioning of the inner catheter distal end proximate to the delivery catheter distal end and manual eversion of the tractor to the inside out configuration along the exterior of the delivery catheter. The procedure is repeated to attempt to withdraw additional thrombus (e.g., thrombus that separated in the previous procedure).

The present subject matter can help provide a solution to these problems, for instance with the thrombus capture and conveyor systems discussed herein. In one example, the system include a conveyor sheath and a conveyor catheter movably received in the conveyor sheath. The system further includes a thrombus conveyor coupled between the conveyor sheath and a conveyor shaft of the conveyor catheter. For instance, the thrombus conveyor includes a conveyor substrate (e.g., mesh, braid, perforated material or the like) having conveyor proximal and distal ends. The conveyor proximal end is coupled with the conveyor sheath, for instance at a conveyor sheath anchor, and the conveyor distal end is coupled with the conveyor shaft with a conveyor shaft anchor. Movement of the conveyor shaft relative to the conveyor sheath causes reversible transitioning of the thrombus conveyor between everted and inverted configurations (e.g., with a conveyor exterior surface facing externally and internally, respectively).

In another example, the system includes a basket catheter movably coupled with the with the conveyor catheter, for instance with a basket shaft of the basket catheter extending through the thrombus conveyor. The basket catheter includes a collection basket coupled with the basket shaft. The collection basket is deployable relative to the basket shaft, for instance to provide a large profile downstream from a thrombus to facilitate capture as described herein.

In operation, the everted thrombus conveyor is deployed from the conveyor sheath and expands to engage the surrounding vessel wall. The conveyor substrate of the thrombus conveyor includes an exterior surface. The portion of the exterior surface that is everted extends between the conveyor sheath and an exterior surface inversion and is engaged along the vessel wall. In an example, the vessel wall obstructs further expansion of the conveyor, and instead the proximal movement of conveyor catheter inverts a portion of the thrombus conveyor at the exterior surface inversion, a fold or joint where the exterior surface transitions from the everted to inverted configurations. The exterior surface inversion corresponds to a conveyor mouth of the thrombus conveyor. The (inverted) portion of the exterior surface that is inverted and extends from the exterior surface inversion to the conveyor shaft is directed inwardly and configured to engage around thrombus.

The collection basket of the basket catheter is deployed distally relative to the thrombus conveyor for instance within or distal to a thrombus. One or more of the thrombus conveyor or the collection basket are moved toward each other to capture the thrombus therebetween. One or more of the thrombus conveyor or the collection basket are moved toward each other until the collection basket and proximal end of the conveyor shaft of the thrombus conveyor are separated by an offset distance. In at least one example, the offset distance corresponds to the uncompressed or minimally compressed length of the thrombus. The offset distance is determined (e.g., by fluoroscopy or the like) or selected by the operator during the operation (e.g., based on haptic feedback, resistance or the like indicating the thrombus is engaged between the basket and the conveyor). In at least one example, the system including one or more the collection basket or the conveyor is configured to tacitly signal the operator (with haptic feedback or the like) when the offset distance corresponds to the length of the thrombus. Arresting relative motion between the basket and the conveyor minimizes longitudinal compression of the thrombus that may otherwise initiate plugging. In one example, the basket catheter is locked to the conveyor shaft, for instance with a catheter clamp such as a nut, clip or the like provided at a proximal location of the system (e.g., outside of the vessels). Locking is initiated with the thrombus captured between the collection basket and the thrombus conveyor (in one example after movement of the basket and the conveyor toward each other). Optionally, the collection basket is locked at the offset distance from the thrombus conveyor corresponding to a length of the captured thrombus.

The conveyor shaft is moved proximally relative to the conveyor sheath. The thrombus conveyor coupled between the conveyor shaft and the conveyor sheath inverts from the everted configuration to an inverted configuration according to this movement. At the same time the collection basket is proximally drawn toward the conveyor sheath by the same movement of the conveyor shaft. The thrombus captured between the collection basket and the thrombus conveyor is guided toward and into the inverting thrombus conveyor, for instance with little or no longitudinal compression of the thrombus to minimize plugging. As the thrombus is received in the thrombus conveyor the proximate conveyor substrate engages with the thrombus and holds the thrombus statically (e.g., there is minimal, including no, relative translating movement between the thrombus and the engaged portion of the conveyor substrate). The statically held thrombus is conveyed (e.g., shuttled in the manner of a conveyor belt) toward the conveyor sheath. The collection basket that moves with the conveyor shaft and the inverted portion of the thrombus conveyor continue to guide thrombus into engagement with the thrombus conveyor. The thrombus conveyor in turn statically couples with the incoming thrombus and guides the received thrombus toward the conveyor sheath.

With continued proximal movement of the conveyor shaft the thrombus conveyor continues to invert and accordingly convey the thrombus therein toward the conveyor sheath. The collection basket is optionally received within the thrombus conveyor and closes the open end of the thrombus conveyor to prevent escape of thrombus. The collection basket, thrombus and the portion of the thrombus conveyor engaged with the thrombus move collectively, for instance with minimal relative translational movement (including none) toward the conveyor sheath. Accordingly, snagging of the thrombus with the conveyor substrate is minimized in comparison to systems that pull or push thrombus relative to a trumpet or sleeve. Instead, the thrombus conveyor is static relative to the thrombus and the conveyor shuttles the thrombus toward the conveyor sheath. Additionally, the thrombus conveyor statically coupled along the thrombus passively compresses or elongates the captured thrombus therein instead of driving the thrombus into a delivery catheter. As shown herein, the conveyor substrate is pliable and complies to the profile of the thrombus. As the thrombus is conveyed toward the conveyor sheath the conveyor passively compresses around the thrombus and elongates the thrombus, and as the thrombus reaches the conveyor sheath the thrombus is passively narrowed and readily fits within the conveyor sheath without plugging. Further, the collection basket guides the thrombus toward the inverting thrombus conveyor (e.g., similar to a plunger) to ensure reception and static engagement of the thrombus along the conveyor substrate.

Additionally, because the thrombus conveyor is coupled between the conveyor sheath and the conveyor shaft the conveyor is readily transitioned from the everted configuration to the inverted configuration and back to the everted configuration. In other words, the thrombus conveyor is reversible. For example, after conveying a first thrombus into the conveyor sheath the thrombus conveyor is readily redeployed from the conveyor sheath. The conveyor shaft is moved distally relative to the conveyor sheath to evert the thrombus conveyor between a sheath distal end of the sheath and a conveyor shaft distal portion of the conveyor shaft (e.g., where the conveyor is coupled). Once everted the conveyor shaft is moved proximally to begin inversion of the thrombus conveyor. Optionally, the collection basket of the collection catheter is deployed again and thrombus is captured between the collection basket and the thrombus conveyor. The thrombus conveyor is transitioned toward the inverted configuration to convey the newly captured thrombus with the conveyor, collectively, toward the conveyor sheath.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a perspective view of a retrieval system.

FIG. 2 is a schematic side view illustrating the locating of a positioning guidewire across a clot within a blood vessel according to an aspect of the present disclosure.

FIG. 3 is a schematic side view illustrating the positioning of a capture/delivery catheter within the blood vessel according to an aspect of the present disclosure.

FIG. 4 is a schematic side view illustrating the positioning of a filter delivery catheter within the blood vessel according to an aspect of the present disclosure.

FIG. 5 is a schematic view illustrating the filter delivery catheter shown in FIG. 4 with the positioning guidewire removed from the blood vessel.

FIG. 6 is a schematic view illustrating the deployment of a filter on a filter guidewire according to an aspect of the present disclosure.

FIG. 7 is a schematic view illustrating the positioning of a capture sleeve sheath according to an aspect of the present disclosure.

FIG. 8 is a schematic view illustrating the deployment of a capture sleeve according to an aspect of the present disclosure.

FIG. 9 is a side view of a retrieval system according to an aspect of the present disclosure.

FIG. 10 is a partial side view of a filter assembly and a capture sleeve of the retrieval system depicted in FIG. 9.

FIG. 11 is a side view of a filter catheter received within a filter delivery catheter of the retrieval system depicted in FIG. 9.

FIG. 12 is a partial side view of the filter catheter depicted in FIG. 11.

FIG. 13 is a cross sectional view of the filter catheter of the retrieval system depicted in FIG. 11.

FIG. 14 is a partial side view of a filter assembly of the filter catheter depicted in FIG. 13.

FIG. 15 is a partial cross-sectional side view of an atraumatic tip of the filter catheter according to an aspect of the present disclosure.

FIG. 16 is a schematic side view illustrating the locating of a positioning guidewire across a clot within a blood vessel according to an aspect of the present disclosure.

FIG. 17 is a schematic side view illustrating the positioning of a capture/delivery catheter within the blood vessel according to an aspect of the present disclosure.

FIG. 18 is a schematic side view illustrating the positioning of a filter delivery catheter within the blood vessel according to an aspect of the present disclosure.

FIG. 19 is a schematic side view illustrating the deployment of at least one filter on a filter catheter according to an aspect of the present disclosure.

FIG. 20 is a schematic side view illustrating the deployed filter catheter depicted in FIG. 19 with the filter delivery catheter pulled axially to expose the filters according to an aspect of the present disclosure.

FIG. 21 is a schematic side view illustrating the positioning of a capture sleeve sheath according to an aspect of the present disclosure.

FIG. 22 is a schematic side view illustrating the deployment of a capture sleeve according to an aspect of the present disclosure.

FIG. 23 is a schematic side view illustrating a retrieval system and an aspiration sheath according to an aspect of the present disclosure.

FIG. 24 is a side view of an aspiration sheath according to an aspect of the present disclosure.

FIG. 25A is a schematic view of a structural layer of an aspiration sheath according to an aspect of the present disclosure.

FIG. 25B is a schematic view of a structural layer of an aspiration sheath according to an aspect of the present disclosure.

FIG. 26 is a partial cross-sectional side view of an aspiration sheath with a dilator according to an aspect of the present disclosure.

FIG. 27A is a schematic side view illustrating a collection catheter and basket catheter retrieving thrombus.

FIG. 27B is a schematic side view of the collection and basket catheters of FIG. 27A with the collection catheter plugged with thrombus.

FIG. 28 is a schematic side view of one example of a thrombus capture and conveyor system.

FIG. 29A is a schematic side view of the thrombus capture and conveyor system of FIG. 28 having a thrombus conveyor in a full everted configuration at deployment.

FIG. 29B is a schematic side view of the thrombus capture and conveyor system of FIG. 28 having the thrombus conveyor in an everted configuration including partial inversion at or after deployment.

FIG. 30 is a detailed side schematic view of the thrombus capture and conveyor system of FIG. 28 illustrating a conveyor mouth profile of the thrombus conveyor.

FIG. 31A is a schematic side view of the thrombus capture and conveyor system of FIG. 28 with thrombus between a collection basket and the thrombus conveyor.

FIG. 31B is a schematic side view of the thrombus capture and conveyor system of FIG. 28 with thrombus captured between the collection basket and the thrombus conveyor.

FIG. 31C is a schematic side view of the thrombus capture and conveyor system of FIG. 28 with the thrombus conveyor in the process of inverting and shuttling thrombus toward a conveyor sheath.

FIG. 31D is a schematic side view of the thrombus capture and conveyor system of FIG. 28 with the thrombus conveyor enveloping the thrombus and the collection basket, and shuttling thrombus toward the conveyor sheath.

FIG. 32A is a schematic side view of another example of a thrombus capture and conveyor system with thrombus between multiple collection baskets and the thrombus conveyor.

FIG. 32B is a schematic side view of the thrombus capture and conveyor system of FIG. 32A with thrombus captured between the collection baskets and the thrombus conveyor.

FIG. 32B′ is a schematic side view of the thrombus capture and conveyor system of FIG. 32A with thrombus separated with the collection baskets.

FIG. 32C is a schematic side view of the thrombus capture and conveyor system of FIG. 32A with the thrombus conveyor in the process of inverting and shuttling thrombus toward the conveyor sheath.

FIG. 33 is a perspective view of an example basket catheter, trumpet catheter and trumpet sheath.

### DETAILED DESCRIPTION

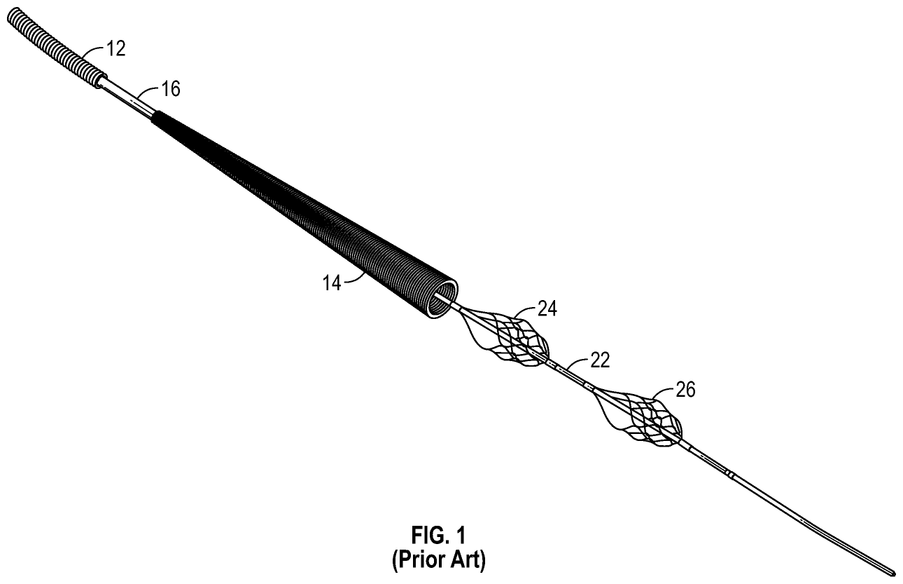

As shown in FIG. 1, the retrieval system 10 can comprise (i) a flexible capture sleeve 14 on the distal end of a flexible sleeve positioning tube 16 and (ii) filters 24, 26 mounted at the distal end of a filter guidewire 22. The retrieval system 10 is shown in U.S. application Ser. No. 12/738,702, which is herein incorporated by reference. The capture sleeve 14 is radially expandable within the blood vessel proximal to the clot. Likewise, the filters 24, 26 are radially expandable within the blood vessel distal to the clot before being pulled through the clot to capture or entrain the clot material. The filters 24, 26 and captured clot are then pulled into the capture sleeve 14, and the capture sleeve 14 encloses the filters 24, 26 and the captured clot material. The capture sleeve 14, filters 24, 26, and the captured clot material are then pulled into a capture/delivery sheath 12 for removal from the body.

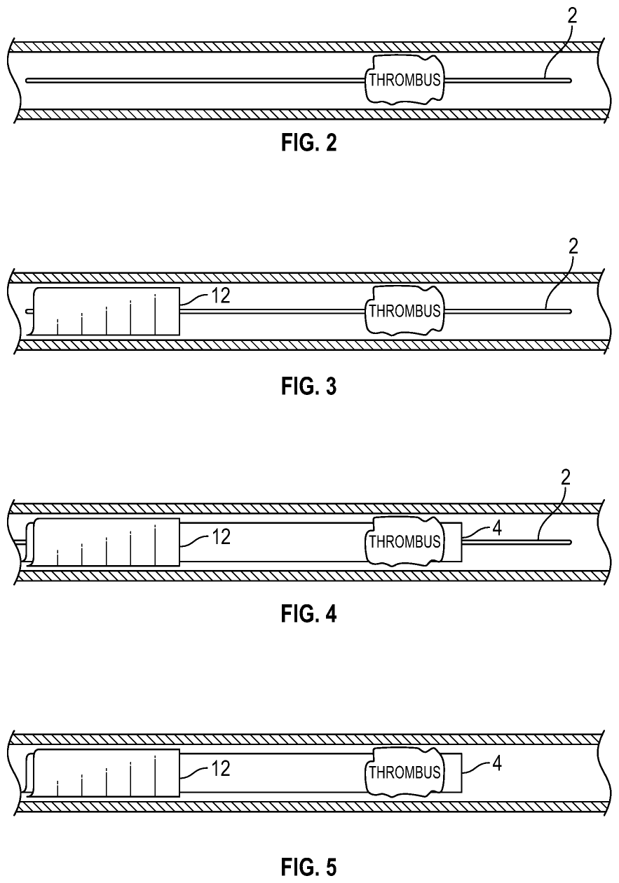

As shown in FIG. 2, the retrieval system 10 is deployed at the clot by initially locating a positioning guidewire 2 and the capture/delivery sheath 12 at the treatment site. The positioning guidewire 2 is located within the blood vessel by navigating the distal end of the positioning guidewire 2 from an access site to the clot. The positioning guidewire 2 is pushed through or around the clot to position the distal end of the positioning guidewire 2 distal to the clot. As shown in FIG. 3, the capture/delivery sheath 12 is guided to the clot over the positioning guidewire 2 to position the distal end of the capture/delivery sheath 12 proximate to the clot. After initial positioning of the capture/delivery sheath 12, the capture/delivery sheath 12 remains in place throughout the procedure including when the filter 24 is redeployed for multiple passes through the clot. However, as discussed further below, the positioning guidewire 2 is removed from the body each time the filters 24, 26, coupled along a filter guidewire 22, are deployed within the blood vessel.

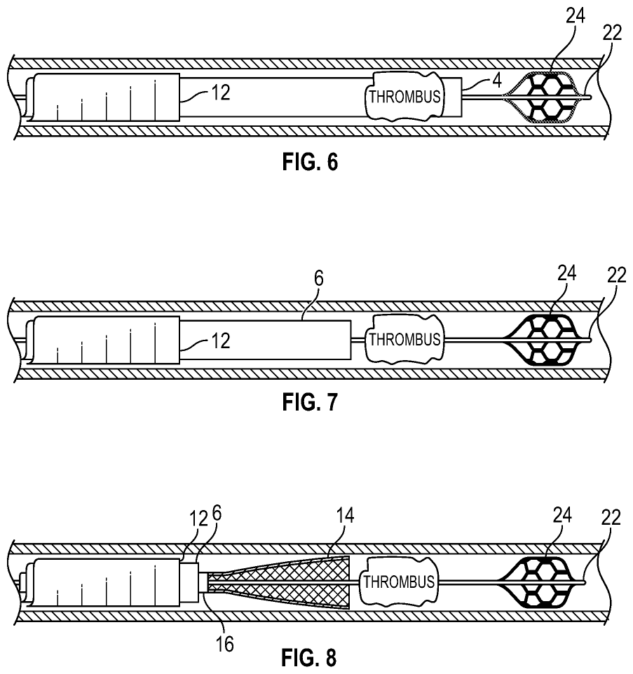

As shown in FIG. 4, the filters 24, 26 are deployed by navigating a filter delivery catheter 4 through the catheter/delivery sheath 12 and over the positioning guidewire 2 to the clot. The distal end of the filter delivery catheter 4 exits the catheter/delivery sheath 12 before continuing over the positioning guidewire 2 across or around the clot. The positioning guidewire 2 serves as a guide for navigating the filter delivery catheter 4 to and past the clot. As shown in FIG. 5, after the filter delivery catheter 4 is positioned, the positioning guidewire 2 is withdrawn from the filter delivery catheter 4 and the body to permit the insertion of a filter guidewire 22 (having the filters 24, 26) through the filter delivery catheter 4. The filter guidewire 22 is then navigated to the clot. As shown in FIG. 6, the filter guidewire 22 is passed through the filter delivery catheter 4 until the filter 24 exits the filter delivery catheter 4 distal to the clot. The filter delivery catheter 4 is sized such that the filter 24 is constrained and collapsed while the filter 24 is moved through the filter delivery catheter 4. The filter 24 self-expands upon exit from the filter delivery catheter 4 once freed from the filter delivery catheter 4. The filter delivery catheter 4 is then withdrawn from the body through the capture/delivery sheath 12.

As shown in FIG. 8, the flexible capture sleeve 14 is deployed proximal to the clot by navigating the capture sleeve 14 on the distal end of the capture sleeve positioning tube 16 to the clot, for instance while within the capture sleeve sheath 6 (see FIG. 7). The flexible capture sleeve 14 is delivered constrained within the distal end of the capture sleeve sheath 6. The capture sleeve sheath 6 is sized to constrain and collapse the flexible capture sleeve 14 when the flexible capture sleeve 14 is held within the capture sleeve sheath 6. The capture sleeve sheath 6 is navigated through the capture delivery sheath 12 until the distal end of the capture sleeve sheath 6 exits the capture/delivery sheath 12 and is positioned proximate to the clot (see FIG. 7). As shown in FIG. 8, the capture sleeve 6 is then slid axially in the proximal direction to uncover the capture sleeve 14 permitting the capture sleeve 14 to self-expand. The filter 24 is then pulled proximally through the clot to engage the clot material and draw the captured clot material into the capture sleeve 14 for removal from the body through the capture/delivery sheath 12.

As discussed above, certain clots may require multiple deployments of filters 24 (or filters 24, 26) distal to the clot and multiple passes of the filters 24, 26 through the clot to fully extract the clot or remove sufficient clot material to restore blood flow through the vessel. After each pass through the clot, the filters 24, 26 and the capture sleeve 14 are removed from the body for cleaning. Each pass of the filters 24, 26 includes a re-deployment of the filters 24, 26 and the capture sleeve 14 on opposing sides of the clot. The filters 24, 26 are collapsed, positioned within the filter delivery catheter 4, and the filter guidewire 22 with collapsed filters 24, 26 are re-delivered to the clot, for instance through the filter delivery catheter 4. To deliver the collapsed filters 24, 26 distal to the clot, the positioning guidewire 2 is first re-inserted and navigated distally past the clot before the filter delivery catheter 4 is navigated past the clot and over the previously navigated positioning guidewire 2.

Re-insertion (and the preceding initial insertion) are conducted carefully to avoid injury to the blood vessel and to consistently position the distal end of the positioning guidewire 2 at a specified location within the blood vessel. However, the clot itself may cause the positioning guidewire 2 to deflect during insertion and each re-insertion as the positioning guidewire 2 engages with the clot while attempting to cross the clot. Accordingly, the clot may frustrate the accurate positioning and consistent repositioning of the positioning guidewire 2 in one or more of the initial insertion or re-insertions. The insertion and reinsertion procedures and attendant positioning of the positioning guidewire 2 (as well as the separate filter delivery guidewire 22 having the filters 24, 26) in each of these procedures add complexity and potentially risky steps to the thrombectomy procedure that are repeated for each pass of the filter 24 (or filters 24, 26) through the clot. As noted herein, each pass of the filter 24 includes withdrawal of the filter 24 and the filter guidewire 22 from the previous pass, re-insertion of the positioning guidewire 22, and re-insertion of the filter guidewire 22 and filter 24 (cleaned). Accordingly, the repeated insertion, re-insertion increases procedure time, is laborious and increases the potential for patient complications.

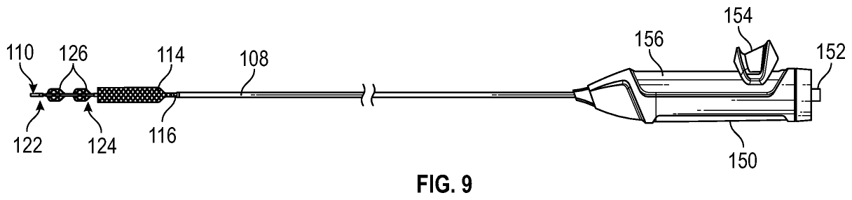

An over-the-wire retrieval system 110 is shown in FIG. 9. The over-the-wire retrieval system 110 includes (i) a flexible capture sleeve 114 coupled with a distal portion of a flexible sleeve positioning tube 116 and (ii) a filter assembly 124 coupled with a filter catheter 122 in contrast to the filter guidewire 22 previously noted above. The filter catheter 122 includes a lumen for slidably receiving a positioning guidewire 102 to permit navigation of the filter catheter 122 along the positioning guidewire 102. Likewise, the flexible sleeve positioning tube 116 defines a lumen for slidably receiving the filter catheter 122 and permits the delivery of the flexible sleeve positioning tube 116 over the filter catheter 122. In this configuration, the filter catheter 122 and the flexible sleeve positioning tube 116 are each delivered and retrieved over the positioning guidewire 102 without removing or repositioning the positioning guidewire 102 from the blood vessel, for instance to permit the delivery of a filter guidewire 22 in the previous example above.

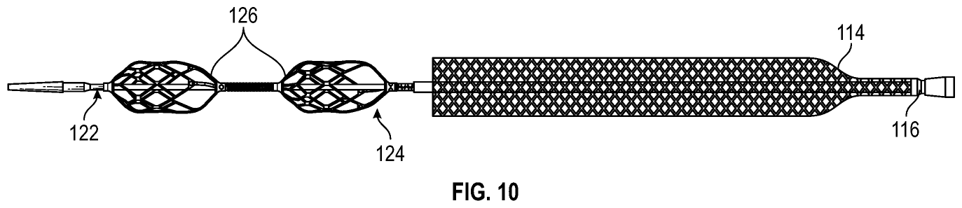

As illustrated in FIG. 10, the capture sleeve 114 in an example includes one or more of a woven material, fenestrated structure, or other filtering structure configured to radially expand (including self-expansion) when unconstrained. In one example, the capture sleeve 114 self-expands within the blood vessel proximate to the clot to fill the vessel or otherwise obstruct the vessel proximal to the clot to prevent the passage of clot while permitting blood flow. The term “clot” used herein and with respect to embodiments of the present invention refers to any clot material within a blood vessel including, but not limited to, one or more of thrombus or emboli; and clot, thrombus and emboli are used interchangeably. Likewise, the filter baskets 126 include a fenestrated structure or other filtering structure configured to radially expand (including self-expansion) when unconstrained to fill or otherwise obstruct the vessel proximal to the clot while permitting blood flow. The term “filter” used here and with respect to embodiments of the present invention refers to woven, braided, fenestrated, or permeable structure capable of mechanically engaging and capturing clot while permitting the flow of blood through the filter baskets 126. As shown in FIG. 9, the filter catheter 122 may comprise multiple filter baskets 126 to enhance clot capture and minimize the loss of clot fragments with each pass. In other embodiments, the filter catheter 122 may comprise a single filter basket 126 to account for complex or tortuous geometry, such as in pulmonary arteries where pulmonary embolisms may become lodged. After deployment of the one or more filter baskets 126, the filter baskets 126 are pulled through the clot to engage the clot material. The one or more filter baskets 126 (with captured clot material) are moved into the capture sleeve 114 to trap the filter baskets 126 and the clot material within the capture sleeve 114. The capture sleeve 114, the one or more filter baskets 126, and the captured clot are then, in one example, pulled into the capture/delivery sheath 112 (see FIG. 18) for removal from the body.

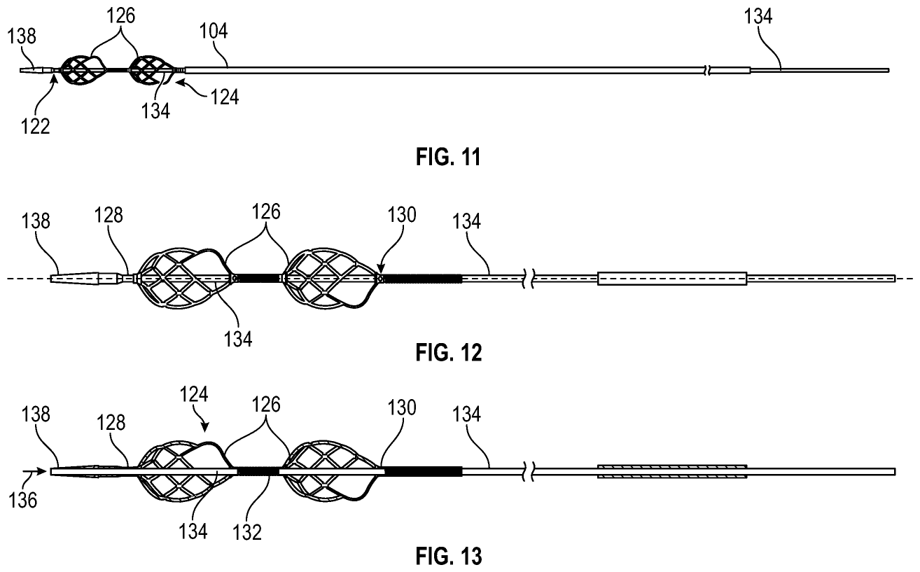

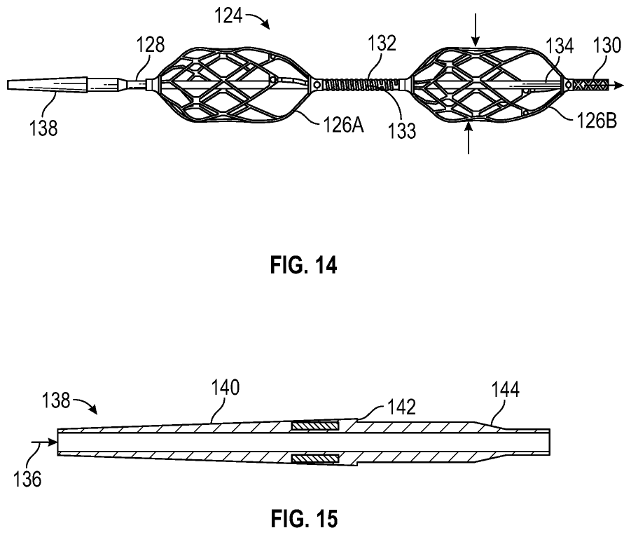

As shown in FIGS. 11-13, the filter catheter 122 includes a catheter shaft 134 defining a central lumen 136 (see FIG. 13). A filter assembly 124 and an atraumatic tip 138 are positioned at the distal end of the catheter shaft 134 (see FIG. 14). As illustrated in FIGS. 11 and 12, a filter delivery catheter 104 can define a central lumen for receiving the catheter shaft 134, which permits translation of the filter delivery catheter 104 axially along the central shaft 130 of the filter catheter 122. The central lumen 136 of the filter catheter 122 is sized to receive a positioning guidewire 102 (See FIG. 16 and later) such that the filter catheter 122 is slidable over the positioning guidewire 102 to the treatment site. The filter catheter 122 is navigated to the treatment site over the positioning guidewire 102, used captures clot, is removed from the body and redeployed without removing or repositioning of the positioning guidewire 102. The positioning guidewire 102 in various examples is between around 50 and 300 cm in length and has a diameter of around 0.014″, 0.018″, or 0.035″ or similar. The positioning guidewire 102 acts as a rail or guide for other components of the retrieval system 110 to translate there along toward the treatment site. The positioning guidewire 102 is slidably received within the central lumen of the catheter shaft 134 of the filter catheter 122. The filter catheter 122 and its catheter shaft 134 optionally serves as a rail or guide for other components of the retrieval system 110. In at least one example, the interior surface, exterior surface, or both surfaces of the catheter shaft 134 of the filter catheter 122 is coated with a lubricious coating to ease sliding movement of the components over the catheter shaft 134.

As shown in FIGS. 13 and 14 the filter assembly 124 includes at least one filter basket 126 having a fixed collar 128 and a sliding collar 130. The fixed collar 128 is, in one example, statically coupled to the catheter shaft 134. The sliding collar 130 is movably coupled to the catheter shaft 134 to permit translational movement of the sliding collar 130 and translational movement of a portion of the filter assembly 124 (e.g., between the collars 128, 130). In this configuration, the sliding collar 130 is translatable axially away from the fixed collar 128 when the filter basket 126 is compressed radially. The translation of the sliding collar 130 thereby permits the one or more filter baskets 126 to collapse radially.

In another example, the filter basket 126 having one or more component baskets is constructed with a shape memory metal, fenestrated structure, or other elastic material that biases the filter basket 126 toward the expanded position (shown FIGS. 13 and 14) while the filter basket 126 is unconstrained, for instance by the capture sleeve sheath 108 (See FIG. 9). When unconstrained (e.g., with the capture sleeve sheath 108 withdrawn) the biased filter basket 126 expands and the sliding collar 130 translates axially toward the fixed collar 128 to permit the radial expansion. In at least one example, at least one of the fixed collar 128 or the sliding collar 130 is notched or etched to form a fenestrated or corrugated structure (e.g., with ridges, knurling, corrugations or the like). The fenestrated structure is, in one example, shaped to increase friction (e.g., by point loading the engagement of the fenestrated or corrugated with the catheter shaft 134) and enhance static coupling between the fixed collar 128 and the catheter shaft 130. Conversely, the fenestrated structure is, in another example, shaped to reduce the contact area between the sliding collar 130 and the catheter shaft 134 to minimize friction between the collar 130 and the shaft 134 and promote translating relative movement.

As depicted in FIGS. 13 and 14, in at least one example, the filter assembly 124 can comprise at least two component filter baskets 126A, 126B connected by an intermediate collar 132. In at least one embodiment, the filter baskets 126A, 126B and intermediate collar 132 include a unitary fenestrated body, such as a laser cut hypotube. The intermediate collar 132 is, in one example, movably coupled along the catheter shaft 134. As shown in FIG. 14 one filter basket (filter 126A as depicted in FIG. 14) includes the fixed collar 128 statically coupled to the catheter shaft 134 and a movable intermediate collar 132 interconnecting the filter basket 126A to the other filter basket 126B. The other basket 126B is movably connected to the catheter shaft 134 with the intermediate collar 132 and a slidable collar 130. The fixed collar 128 of filter basket 126A positions both filter baskets 126A, 126B for radial expansion and compression proximate a distal end portion of the catheter shaft 134 while the slidable intermediate collar 132 and the slidable collar 130 permits movement of both filter baskets 126A, 126B to radially compress and expand.

In another example, the static coupling provided with the fixed collar 128 braces the one or more filter baskets 126A, B during translations of the baskets 126A, B, for instance into the capture sleeve 114. As the catheter shaft 134 of the filter assembly 124 is drawn proximally, the filter baskets 126A, B are correspondingly drawn proximally. The filter baskets 126A, B engage with clot material and continued proximal movement of the catheter shaft 134 pulls the baskets and clot material into the capture sleeve 114. The movable couplings provided with intermediate collar 132 and the slidable collar 130 permits axial compression of the filter assembly 124 and its baskets 126A, B toward the static fixed collar 128. The baskets 126A, B accordingly compress with proximal movement of the catheter shaft, the associated elements of the baskets (e.g., wires, filaments, mesh or the like) aggregate over each other, and fenestration openings effectively shrink. The capture of the clot material is accordingly enhanced with the aggregated elements of the baskets 126A, B. Further, with additional proximal movement of the catheter shaft the filter assembly 124 compresses until gaps in the intermediate collar (described herein below), and fenestration openings in the baskets 126A, B or the like, are compressed and the material of the baskets 126A, B and the intermediate collar 132 stacks (e.g., a compression limit). The fixed collar 128 braces the baskets 126A, B and the intermediate collar 132 against further movement and instead transmits the proximal movement of the catheter shaft 134 fully to the baskets and drives the baskets into the capture sleeve 114.

As illustrated in FIG. 13, the fixed (distal) collar 128 is fixed to the catheter shaft 134 while the slidable (proximal) collar 130 and the intermediate collar 132 are movable along the catheter shaft 134. In various embodiments, different combinations of collars 128, 130, 132 are movable or static relative to the catheter shaft 134. For instance, in one example, the distal collar 128 and intermediate collar 132 are movable over the catheter shaft 134, and the proximal collar 130 is statically coupled with the catheter shaft 134. In another example, the intermediate collar 132 is statically coupled on the catheter shaft 134 while the distal collar 128 and the proximal collar 130 are movably coupled along the catheter shaft 134.

In at least one example, the intermediate collar 132 includes notches 133 (also referred to as gaps, openings or the like) that permit axial expansion or compression of the intermediate collar 132. The permitted expansion or compression provides a deflectable quality (e.g., pliability, compressibility or the like) to intermediate collar 132 and permits it to behave similar to a spring between filter baskets 126A, 126B. The intermediate collar 132 also allows filter baskets 126A, 126B to move relative to each other to absorb and minimize stress that may cause damage to the filter assembly 124 as the filter assembly 124 is pulled through the clot. Additionally, the relative movement permits the application of a consistent force on the catheter shaft 134 while minimizing one or both of sudden engagement with thrombus and seizing of the catheter and sudden disengagement of the filter baskets 126A, B from thrombus. Instead, the intermediate collar 132 provides compression (as well as expansion) that permits the gradual escalation of force from the catheter shaft 134 to the filter baskets 126A, B and the associated thrombus during engagement and after engagement (e.g., when the thrombus is pulled). As noted above, the intermediate collar 132 and the filter baskets 126A, B optionally axially compress during capture of clot material and proximal movement in a manner that aggregates elements of the filter baskets 126A, B and enhances capture and driving of the clot material into the capture sleeve 114. In another example, the filter baskets 126A, 126B simultaneously engage different portions of the clot material as the filter baskets 126A, 126B are pulled through the clot and engage clot material. For example, one filter basket 126A (or a portion of the basket 126A) engages more organized and difficult to move thrombus material while filter basket 126B (or a portion thereof) engages less organized and easier to move thrombus material. The flexibility provided with the intermediate collar 132 (e.g., as a biasing element, shock absorber, damping element or the like), as well as the deformability of the baskets 126A, B themselves, modulates the application of force to each type of thrombus in a manner that permits retrieval of thrombus having thrombus material with a range of consistencies without sudden engagement and disengagement.

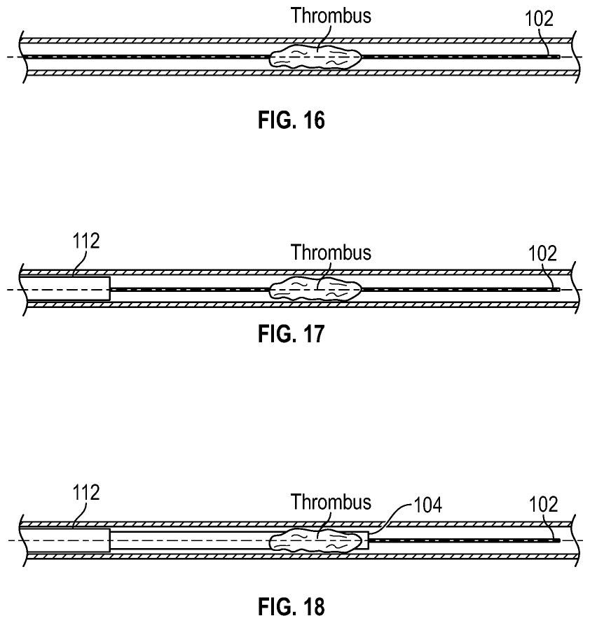

As illustrated in FIG. 15, the atraumatic tip 138 can comprise a front sloped portion 140. The front sloped portion 140 can be angled to ease insertion of the filter catheter 122 into the capture/delivery sheath 112 and to decrease engagement or snagging of these structures, for instance as the filter catheter 122 is loaded into the capture/delivery sheath 112. The shape of the atraumatic tip 138 eases navigation of the filter catheter 122 across the thrombus. In at least one embodiment, the outer diameter of the front sloped portion 140 (e.g., a taper) corresponds to the outer diameter of the filter delivery catheter 104 (See FIG. 11). The filter delivery catheter 104 is slid axially over the filter catheter 122 until the sloped portion 140 protrudes from the distal end of the filter delivery catheter 104 and the remainder of the filter catheter 122 is concealed within the catheter 104. In this configuration, the atraumatic tip 138 operates as a dilator for the filter delivery catheter 140 and facilitates navigation and delivery of the filter delivery catheter 104 and the filter catheter 122 to the treatment site and across the thrombus together. As further shown in FIG. 15, the atraumatic tip 138 optionally includes a shoulder 142 for engaging the distal end of the filter delivery catheter 104, and in one example provides a flush (including near flush) interface between the catheters 104, 122. In another example, the atraumatic tip 138 includes a rear sloped portion 144 (e.g., a taper) for aligning and guiding the distal end of the filter delivery catheter 104 into engagement with the shoulder 142.

As illustrated in FIG. 9, in at least one embodiment, the over-the-wire retrieval system (OTW retrieval system) 110 includes a handle 150 for operating the OTW retrieval system 110. The proximal end of the capture sleeve positioning tube 116 is statically coupled to the proximal end of the handle 150 and extends through the handle 150 to provide a central lumen through the handle 150. In this configuration, the handle 150 includes a seal or Luer connector 152 for controlling access to the proximal end of the capture sleeve positioning tube 116. The capture sleeve positioning tube 116 is coaxially positioned within the capture sleeve sheath 108 wherein the proximal end of the capture sleeve sheath 108 is received within the handle 150. The handle 150 shown in FIG. 9 includes an operator, such as a slider 154, attached to the proximal end of the capture sleeve sheath 108. The slider 154 is moveable (e.g., axially in this example) to move the capture sleeve sheath 108 axially relative to the capture sleeve positioning tube 116. In at least one example, the handle 150 includes a track 156 coupled with the slider 154, and the track 156 guides movement of the slider 154. Optionally, the handle 150 includes a locking mechanism that biases the slider 154 toward a secure position that correspondingly secures the associated capture sleeve sheath 108 in position. Accordingly, the operator moves the capture sleeve sheath 108 with operation of the slider 154, and upon release of the slider 154 the capture sleeve sheath 108 remains in a static position (e.g., relative to the capture sleeve positioning tube 116).

FIGS. 16-24 illustrate operation of the OTW retrieval system 110 for retrieval of thrombus (e.g., a clot). As shown in FIG. 16, a positioning guidewire 102 is navigated through a blood vessel from an access site toward a treatment site, for instance having the thrombus. The positing guidewire 102 is pushed across or around the thrombus to position the distal end of the positioning guidewire 102 distal relative to the thrombus. The positioning guidewire 2 operates as a rail or guide for navigating the various components of the OTW retrieval system 110 proximate to the thrombus (e.g., proximal, distal and through or around the thrombus). The positioning guidewire 102 is, in one example, conventionally dimensioned, for instance with a diameter of between around 0.018″ to 0.035″ and a length between around 50 to 260 cm. In certain embodiments, the positioning guidewire 102 includes a hydrophilic coating for easing navigation of the positioning guidewire 102 through the blood vessel and to facilitate the movement of components of the retrieval system 110 over the positioning guidewire 102.

As depicted in FIG. 16, the positioning guidewire 102 is navigated through the thrombus. In another example, the positioning guidewire is navigated through the thrombus or between the thrombus and the vessel wall, for instance depending on the density of the thrombus. The distal end of the positioning guidewire 102 extends a specified distance past the thrombus to provide a runway for deployment of various components of the OTW retrieval system 110 distal to the thrombus. Additionally, the positioning of the distal end of the positioning guidewire 102 secures the positioning guidewire 102 in place and minimizes inadvertent withdrawal of the guidewire 102 through the thrombus (e.g., proximally). Unlike the other retrieval system 10 discussed herein having the guidewire 2 that is removed and re-inserted in each pass, the positioning guidewire 102 for the current OTW retrieval system 110 remains in place for the procedure including potential multiple passes of the filter delivery catheter 104.

As shown in FIG. 17, the capture/delivery sheath 112 is guided through the vessel to the thrombus over the positioning guidewire 102. The distal end of the capture/delivery sheath 112 is positioned proximal to the thrombus (e.g., a specified distances to permit deployment of one or more other features of the system 110). Once positioned, the components of the retrieval system 110 are navigated through the vessel and within the lumen of the capture/delivery sheath 112 to the thrombus over the positioning guidewire 102. As shown in FIG. 17, the distal end of the capture/delivery sheath 112 is proximally spaced from the thrombus to permit deployment of various components of the OTW retrieval system 110 between the thrombus and the distal end of the capture/delivery sheath 112. After the capture/delivery sheath 112 is positioned, the capture/delivery sheath 112 optionally remains in place with the positioning guidewire 102 throughout the procedure for navigating and retrieving the various components of the OTW retrieval system 110 to and from the treatment site within the vessel. Accordingly, the positioning guidewire 102 and the capture/delivery sheath 112 are configured to provide inner and outer guidance of components to the treatment site; inner with components coupled over the positioning guidewire 102 and outer with the capture/delivery sheath 112 surrounding the components.

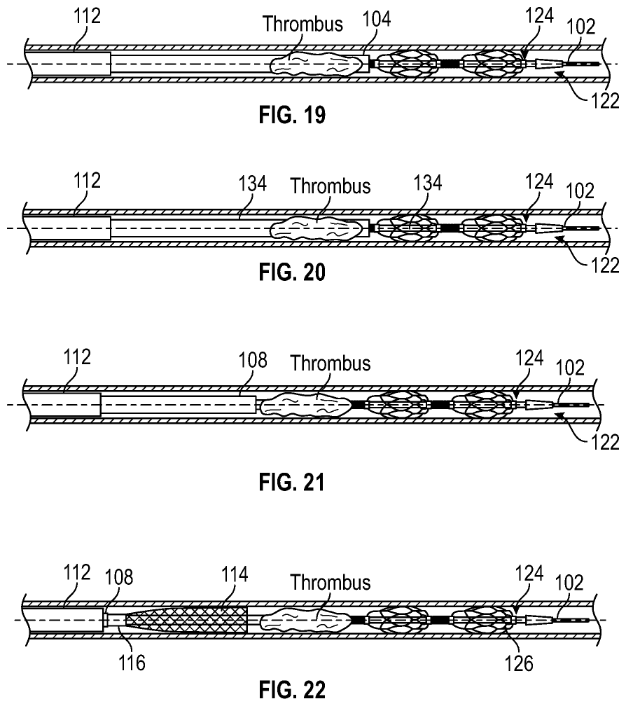

As shown in FIG. 18, the filter delivery catheter 104 is guided over the positioning guidewire 102 and through the catheter/delivery sheath 112. The filter delivery catheter 104 exits the catheter/delivery sheath 112 proximate to the thrombus in FIG. 19. The filter delivery catheter 104 is further advanced axially over the positioning guidewire 102. As shown in FIG. 19 the distal end of the filter delivery catheter 104 is positioned distal to the thrombus. In some examples thrombus may be more organized, dense or the like and accordingly more difficult to cross. The positioning guidewire 102 maintains the general alignment of the filter delivery catheter 104 with the vessel walls and minimizes the deflection of the filter delivery catheter 104 as the filter delivery catheter 104 is pushed across the thrombus. For example, the positioning guidewire 102 provides guidance to the delivery catheter 104 to ensure delivery through the vessel in contrast to engagement with the vessel wall.

As shown in FIG. 19, the filter catheter 122 includes a central lumen for receiving the positioning guidewire 102. The filter catheter 122 is navigated over the positioning guidewire 102 and through the filter delivery catheter 104 to the treatment site, for instance distal to the thrombus as shown. The filter catheter 122 includes a filter assembly 124 positioned proximate to a distal end of the filter catheter 122. The filter catheter 122 is navigated through the filter delivery catheter 104 until the filter assembly 124 exits the filter delivery catheter 104. The filter assembly 124 comprises at least one filter basket 126 (two component filter baskets are shown in FIG. 19) configured to expand (including self-expand) when unconstrained by the filter delivery catheter 104. As shown in FIG. 19 the example filter baskets 126 self-expand upon exiting the filter delivery catheter 104.

As shown in FIGS. 19 and 20, the filter delivery catheter 104 is in one example pulled proximally across the thrombus to uncover the filter baskets 126 and the catheter shaft 134 of the filter catheter 122. The filter baskets 126 are accordingly deployed distal to the thrombus to facilitate capture and retraction of the thrombus into the capture/delivery sheath 112. As discussed herein, the filter baskets 126 are withdrawn into the capture/delivery sheath 112 when withdrawal of the system 110 is specified. In at least one embodiment, the filter delivery catheter 104 is fully withdrawn from the body through the capture/delivery sheath 112, for instance to provide space within the central lumen of the capture/delivery sheath 112 for the delivery of various other components of the OTW retrieval system 110 and for the withdrawal of thrombus. In at least one embodiment, the filter delivery catheter 104 is retracted proximally to deploy the filter baskets 126 but is not otherwise retracted from the body. In this configuration, the filter delivery catheter 104 is optionally moved forward (relative to the baskets 126) in the distal direction to constrain and collapse the filter baskets 126, for instance for repositioning of the baskets 126. In another example, the filter baskets 126 are moved proximally to constrain and collapse the filter baskets 126.

As shown in FIG. 21, after the filter delivery catheter 104 is optionally withdrawn a capture sleeve sheath 108 is navigated over the shaft of the filter delivery catheter 104 and through the capture/delivery sheath 112 to the treatment site. The distal end of the capture sleeve sheath 108 is navigated distally until the distal end of the capture sleeve sheath 108 exits the capture/delivery sheath 112 and is positioned proximate to the thrombus. As illustrated in FIG. 21, a distal end portion of the capture sleeve sheath 108 is positioned adjacent to the proximal side of the thrombus. A capture sleeve 114 is attached to the distal end of a capture sleeve positioning tube 116 nested within the capture sleeve sheath 108. The capture sleeve positioning tube 116 is navigated over the shaft of the filter delivery catheter 104 and through the capture sleeve sheath 108 to the treatment site. In certain embodiments, the capture sleeve positioning tube 116 and the capture sheath 108 are navigated to the treatment site. In this configuration, during navigation, the capture sleeve 114 is positioned within the distal end of the capture sheath 108 such that the capture sheath 108 maintains the capture sleeve 114 in a collapsed configuration during navigation of both devices.

As shown in FIG. 22, the capture sleeve sheath 108 is pulled in the proximal direction to expose the capture sleeve 114 and deploy the capture sleeve 114. The capture sleeve 114 is, in one example, configured to self-expand when exposed by the proximal movement of the capture sleeve sheath 108, for instance with shape memory materials, return bias provided by the previously constrained capture sleeve 114 or the like. The capture sleeve 114 is configured to at least partially fill the vessel while permitting blood flow and receiving thrombus engaged by the at least one filter 126 and driven toward the sleeve 114. The filter assembly 124 including the filters 126 is pulled proximally to engage at least a portion of the thrombus with the one or more filters 126 and pull the thrombus into the deployed capture sleeve 114. Once the thrombus is pulled into the capture sleeve 114, the capture sleeve 114 (with captured thrombus and the one or more filters 126 therein) are pulled into the capture/delivery sheath 112 and withdrawn from the body.

In certain situations, the filter 126 can miss the thrombus or pulls a small portion of the overall thrombus into the one or more filters 126. In this arrangement, the filter delivery catheter 104 (See FIGS. 18 and 19) is optionally moved distally over the one or more filters 126 to “re-sheath” the filters 126 including collapse of the filters 126 with the filter delivery catheter 104 after the filters 126 are within the capture sleeve 114. Thrombus material already entrained in the one or more filters 126 is macerated or wrung from the filters 126 as the filters 126 are collapsed by the filter delivery catheter 104. The filter delivery catheter 104 and the re-sheathed filter 126 are navigated over the positioning guidewire 102, past the thrombus, for another pass to capture additional thrombus. Re-sheathing and re-deployment and capture of additional thrombus are conducted without fully removing and re-deploying one or more components of the OTW retrieval system 110, for instance, the positioning guidewire 102, the filter delivery catheter 124 or the like. Instead, these components remain proximate to the thrombus, and are reset (e.g., re-sheathed) for immediate use to capture and withdraw additional thrombus.

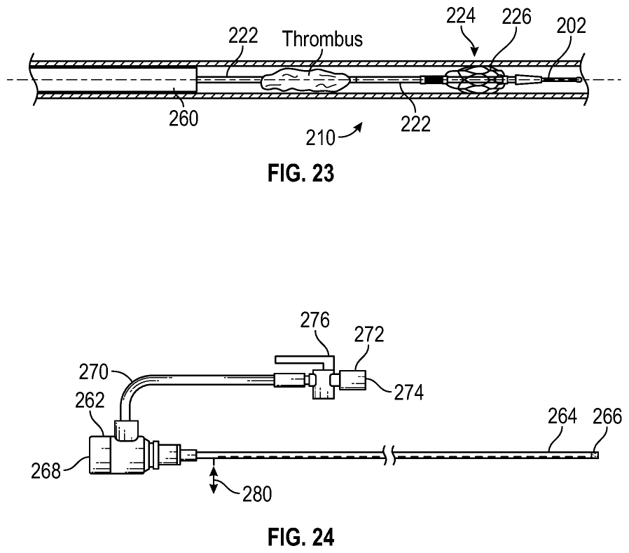

Another example OTW retrieval system 210 is shown in FIG. 23. The OTW retrieval system 210 includes a filter assembly 224 mounted on a filter catheter 222 and (ii) an aspiration sheath 260. In this configuration, the filter assembly 224 includes at least one filter 226. As shown, the at least one filter 226 is deployed distal to the illustrated thrombus, for instance with retraction of a filter delivery catheter (e.g., similar to the filter delivery catheter 104 shown in FIG. 19). The at least one filter 226 is configured to expand within a vessel distal to the thrombus to fill the vessel and prevent passage of clot material past the filter 226. The at least one filter 226 is fenestrated and permits blood flow therethrough. The expanded filter assembly 224 is pulled toward the thrombus to engage thrombus and draw the thrombus toward the mouth of the aspiration sheath 260. A negative pressure (e.g., vacuum) is supplied by the aspiration sheath 260 at its open distal end to aspirate the clot material into the aspiration sheath 260. Accordingly, the aspiration sheath 260 ‘cleans’ thrombus from the at least one filter 226 and, as noted herein, prepares the at least one filter 226 for re-deployment and capture of additional thrombus. In one example, the positioning of the thrombus proximate to the opening of the aspiration sheath 260 permits the application of a lesser negative pressure because of the thrombus proximity. This minimizes the aspiration of blood, body fluids or the like and minimizes vacuum engagement of the aspiration sheath 260 with the vessel wall. In contrast, other aspiration systems apply greater negative pressure to aspirate thrombus material remote to an aspiration catheter opening and unintentionally withdrawn additional blood and body fluids.

As shown in FIG. 23, the filter catheter 222 includes one or more filter baskets 226 having a fenestrated structure or other structure that permits blood flow while capture thrombus. The filter catheter 222 biases the one or more filter baskets 226 to radially deploy when unconstrained. For instance, the one or more filter baskets 226 are configured to self-expand from a constrained configuration during delivery to a deployed configuration when unconstrained (e.g., with retraction of a filter delivery catheter). As illustrated in FIG. 23, in at least one embodiment, the filter catheter 222 includes at least one filter basket 226. To accommodate the geometry of pulmonary vessels, the filter basket 226 includes a complementary geometry, including, but not limited to, a spherical geometry, cylindrical geometry or the like as depicted in FIG. 23 when expanded. The one or more filter baskets 226 are deformable and conform to variations in pulmonary vessel walls to fit within and consistently engage along vessel walls. In certain embodiments, the filter catheter 222 comprises a plurality of filter baskets 226 arranged axially along the filter catheter 222.

The filter catheter 222, similar to the filter catheter 122, includes a lumen for movably receiving a positioning guidewire 202 for delivery and retrieval of the filter catheter 222 over the positioning guidewire 202. The filter catheter 222 is navigated over the positioning guidewire 202 to the thrombus and across the thrombus to position the filter assembly 224 distal to the thrombus. The filter catheter 222 is optionally conveyed within a filter delivery catheter (e.g., see filter delivery catheter 104 in FIGS. 18 and 19). The filter delivery catheter constrains the at least one filter basket 226 of the filter assembly 224 in a collapsed configuration for delivery. Once the collapsed filter basket 226 is positioned distal to the thrombus, the filter delivery catheter is retracted to uncover the filter basket 226, and the filter basket 226 expands radially distal to the thrombus. In certain embodiments, the one or more filter baskets 226 are configured for re-sheathing in situ. For instance, the filter delivery catheter 204 is moved over the deployed one or more filter baskets 226 to constrain and collapse the filter baskets 226. The re-sheathed and collapsed one or more filter baskets 226 are moved distal to the thrombus (e.g., over the positioning guidewire 202) for redeployment of the filter baskets 226 distal to the clot for a supplemental pass (or passes) through the thrombus. The re-sheathing permits resetting of the one or more filter baskets 226 for rapid redeployment proximate to the thrombus. For example, re-sheathing permits rapid multiples passes of the filter basket 226 through the thrombus and over the positioning guidewire 202. In operation, the one or more filter baskets 226 quickly “feeds” thrombus material to the opening of the aspiration sheath 260 for aspiration and removal from the blood vessel. The suction applied through the aspiration sheath 260 and available at the opening of the sheath permits the aspiration sheath 260 to continually extract thrombus as the thrombus material is brought to the opening of the aspiration sheath 260 by the one or more filter baskets 226. The aspiration sheath 260 ‘cleans’ the one or more filter baskets 226, and optionally the OTW retrieval system 210 is operated to re-sheath the filter baskets 226 for redeployment proximate to the thrombus, capture of additional thrombus and repeated aspiration or ‘cleaning’ of the thrombus from the filter baskets 226.

As shown in FIG. 24, the aspiration sheath 260 includes, in an example, a main hub 262 and a sheath body 264 that extends distally from the main hub 264. The sheath body 264 includes a lumen 266 accessible through the main hub 262. The sheath body 264 optionally includes a secondary lumen for the delivery of diagnostic or therapeutic agents to the treatment site separate from the lumen 266. In one example, the lumen 266 is configured for receipt of one or more components of the OTW retrieval systems discussed herein, and for capture and removal of thrombus. In at least one example, the interior surfaces, exterior surfaces, or both surfaces the sheath body 264 are coated with a lubricious coating to ease sliding movement of components through the sheath body 264 and through vasculature. In at least one embodiment, the main hub 264 includes a hemostasis valve 268 to seal the proximal end of the lumen 266 of the sheath body 262. In an embodiment, the aspiration sheath 260 includes a side path 270 extending from a secondary hub 272 to the main hub 264. The side path 270 includes side lumen 274 accessible through the secondary hub 272. The distal end of the side lumen 274 of the side path 270 is fluidly connected to the lumen 266 of the sheath body 264. In at least one embodiment, a syringe is connected to the secondary hub 272 to apply a negative pressure through the side lumen 274 of the side path 270 and the lumen 266 of the sheath body 264. The applied negative pressure draws thrombus material into the distal opening of the sheath body 264 as discussed herein. The side path 270 optionally includes a manual valve 276 that selectively controls the application of negative pressure through the side lumen 274 of the side path 270 and correspondingly the lumen 266 of the sheath body 264.

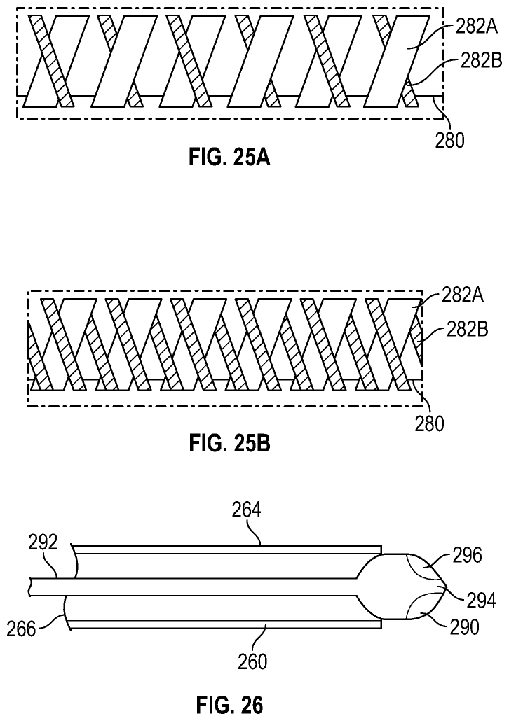

As shown in FIGS. 25A-B, the sheath body 264 of the aspiration sheath 260 optionally includes a braided structural layer to provide flexibility and pushability for the sheath body 264. As illustrated in FIGS. 25A-B, the braided structural layer includes at least a first filar array 282A and a second filar array 282B. The first filar array 282A is interwoven into the braided layer such that the filars of the first filar array 282A are arranged in a first direction while the second filar array 282B is interwoven into the braided layer such that the filars of the second filar array 282B are arranged in a second direction transverse to the first direction (e.g., at an angle to the first direction). In an example, the first filar array 282A includes at least one first braid wire have a cross-section of between around 0.003″×0.007″ to around 0.005″×0.015″. The second filar array 282B includes at least one second braid wire having a cross-section of between around 0.001″×0.003″ to around 0.003″×0.008″. In an example, the first filar array 282A and the second filar array 282B interwoven in an alternating 1:1 pattern as shown FIG. 25A. In another example, the first filar array 268A and the second filar array 282B are interwoven in an unbalanced fashion as shown FIG. 25B, for instance having greater number of filars in the second filar array 282B or a higher density (e.g., pics per inch) of filars. The ratio of filars between first and second filar arrays 282A, B include, but are not limited to 1:2, 1:3, 1:4, 2:3, 2:4, 2:5 and so on.

As shown in FIGS. 24 and 25A-B, in at least one embodiment, the aspiration sheath 260 includes a pull wire 280 anchored to the distal end of the sheath body 264 and extending toward the proximal end of the sheath body 264. In operation, the pull wire 280 is tensioned (e.g., from distal to proximal) to deflect the distal end of the sheath body 264. Actuation of the pull wire 280 angles the distal end of the sheath body 264, for instance to navigate the sheath body 264. In other examples, angling of the distal end of the sheath body enhances the direction of aspiration through the opening of the aspiration sheath 260, for instance toward collected thrombus material that has accumulated along one or more sides of the filter baskets 126, 226. In at least one embodiment, the distal end of the sheath body 264 includes a plurality of slits or relief cuts that enhance tip compliance and flexibility and thereby enhance direction of the aspiration catheter 260 with actuation of the pull wire 280. Optionally, the pull wire 280 is braced within the aspiration catheter 260 and axial loading (e.g., compression toward the distal end) of the pull write 280 permits actuation in a different orientation relative to tension.

As shown in FIG. 26, in at least one embodiment, the aspiration sheath 260 is delivered to the thrombus over a guidewire (such as the positioning guidewire 102) or over the shaft of the filter catheter 222. In one example a dilator 290 is inserted through the aspiration sheath 260, and as shown in FIG. 26, a dilator head 292 at least partially protrudes from the mouth of the aspiration sheath 260. The dilator includes the dilator head 292 and a shaft 294 coupled with the dilator head 292. As illustrated in FIG. 26, the dilator head 292 is optionally tapered from an outer diameter approximating the opening of the sheath body 264 to a smaller diameter (e.g., the dilator head 292 is tapered). In one embodiment, the dilator head 292 includes one or more relief scallops 296 (e.g., cuts, grooves, corrugations or the like) that permit fluid to flow past the dilator head 292 and into the aspiration sheath 260. The dilator head 292 and the one or more relief scallops 296 minimize vacuum sealing between the dilator head 292 and the aspiration sheath 260, and minimize air entrainment or air bubble retention in the aspiration sheath 260 for example as the dilator head 292 is moved through the lumen 266 of the sheath body 264 and the lumen 266 is at least partially filled with blood or saline. The inner diameter of the lumen 266 approximates the outer diameter of the dilator head 292 to minimize the edge of the sheath body 264 standing proud relative to the dilator head 292. In another example, the tapered profile of the dilator head 292 minimizes the stepped interface between the dilator head 292 and the sheath body 264 and accordingly provides for a flush (including near flush) transitions that minimizes snagging of vasculature, thrombus or the like.

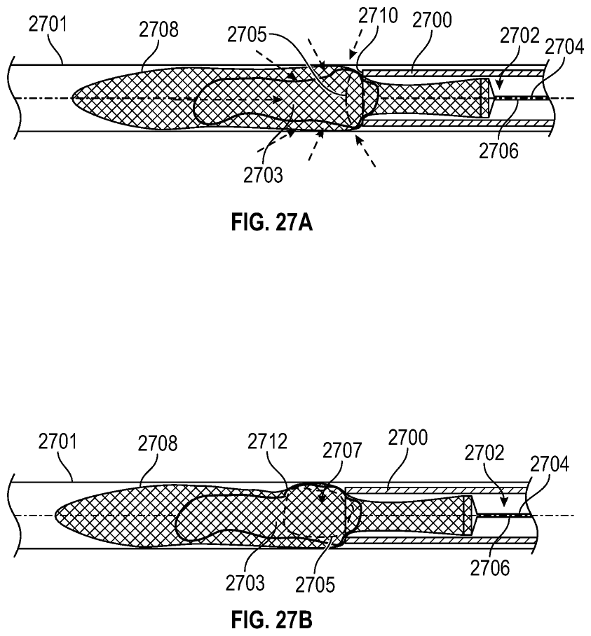

FIGS. 27A and 27B show another example of a collection catheter 2700 and a basket catheter 2704 in a vessel 2701. The basket catheter 2704 includes a basket 2708 coupled with a casket shaft 2706, and the basket shaft is slidably received within a catheter lumen 2702 of the collection catheter 2700. As shown in FIG. 27A, a portion of the basket 2708 is distal to a catheter mouth 2710 of the collection catheter 2700, and the thrombus 2703 is within the basket 2708.

The thrombus 2703 may, in some examples, have varied consistencies, density or the like. Some portions have a gelatinous consistency, other portions are fibrous and have an organized consistency, and still other portions have consistencies therebetween. Thrombus 2703 in some examples has mixed consistencies and accordingly behaves in sometimes unpredictable fashions.

The basket catheter 2704 is proximally moved relative to the collection catheter 2700. The relative movement of the basket 2708 pulls the basket and the thrombus 2703 toward and into the catheter mouth 2710. As shown in FIG. 27A, the thrombus 2703 has begun to lodge within the catheter mouth 2710 and is forming a thrombus plug 2705. Continued movement of the basket catheter 2704 with the basket shaft 2706 pulled relative to the collection catheter 2700 drives the thrombus 2703 toward the catheter mouth 2710, see FIG. 27B. The thrombus plug 2705 resists the proximal movement, and instead the thrombus 2703 aggregates proximate to the catheter mouth 2710. As shown in FIG. 27B, the thrombus plug 2705 enlarges with the proximal movement of the basket catheter 2704 into the collection catheter 2700. The enlarged thrombus plug 2705 aggravates the movement of the thrombus 2703 into the collection catheter 2700.

As further shown in FIG. 27B, the thrombus plug 2705 in some examples binds with the basket 2708 of the basket catheter 2704. Accordingly, as the basket 2708 is proximally pulled into the collection catheter 2700 the thrombus plug 2705 contorts, deforms or the like and the bound basket 2708 (e.g., snagged with the thrombus 2703) similarly contorts or deforms. In the example shown in FIG. 27B at a snagged basket portion 2712 of the basket 2708 has deformed and wrapped around the thrombus plug 2705. The binding of the basket 2708 and the thrombus 2703 further aggravates the plugging of the catheter mouth 2710 and thereby aggravates retraction of the thrombus 2703 into the collection catheter 2700 (e.g., the catheter lumen 2702).

In one example, a technician may attempt to pull the basket catheter 2704 to overcome the thrombus plug 2705 and the potentially snagged basket portion 2712. Increased proximal force, in some examples, further plugs the thrombus 2703 proximate to the catheter mouth 2710. Additionally, snagging of the of collection basket 2708 is further aggravated in some examples. In other examples proximal movement may pull the thrombus 2703, at least partially, into the collection catheter lumen 2702. However, the relative movement between the thrombus 2703 and basket 2708 relative to the catheter mouth 2710 may shear thrombus particulate from the thrombus 2703 (with the catheter mouth) and further complicate the procedure (e.g., require lytic infusions, additional retrieval procedures or the like). The proximal movement may, in some examples, damage one or more of the basket catheter 2704, the collection catheter 2700, or both and frustrate performance of the procedure. If the thrombus 2703 is not withdrawn, and the collection catheter 2700 and the basket catheter 2704 are not readily recoverable a procedure, such as a venous cutdown procedure, is conducted to surgically open the vessel 2701 and retrieve the catheters 2700, 2704.

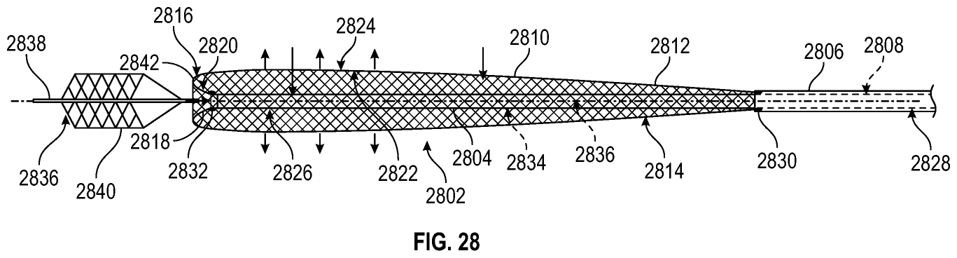

FIG. 28 illustrates an example of a thrombus capture and conveyor system 2800 that minimizes relative movement between the features of the system that engage with, capture, and withdraw thrombus from a vessel. Instead, the thrombus capture and conveyor system 2800 gathers thrombus proximate to a conveyor mouth 2818 of an everted thrombus conveyor 2810. Inversion of the thrombus conveyor 2810 shuttles thrombus in the manner of a conveyor carrying materials (e.g., the material is static relative to the conveyor, and moves with the conveyor) toward and into a conveyor sheath 2806. The thrombus is statically conveyed or shuttled with the thrombus conveyor 2810 and passively narrowed, thinned or the like as the thrombus conveyor 2810 travels toward the conveyor sheath 2806. At the conveyor sheath 2806 the thrombus and thrombus conveyor 2810 (static relative to the thrombus) are readily moved into the sheath without relative movement that causes plugging, snagging or the like in other systems.

As shown in FIG. 28, the thrombus capture and conveyor system 2800 includes a conveyor sheath 2806 having a sheath lumen 2808 as part of a conveyor catheter 2802. The conveyor catheter 2802 includes a conveyor shaft 2804 movably received within the sheath lumen 2808 of the conveyor sheath. As described herein, the thrombus conveyor 2810 is coupled with each of the conveyor shaft 2804 and the conveyor sheath 2806 of the conveyor catheter 2802. For instance, as shown in FIG. 28, a conveyor proximal end 2814 of the thrombus conveyor 2810 is coupled (welded, crimped, adhered or the like) at a conveyor sheath anchor 2830 of the conveyor sheath 2806. A conveyor distal end 2816 of the thrombus conveyor 2810 is coupled with the conveyor shaft distal portion 2826, for instance with a conveyor shaft anchor 2832.

The thrombus conveyor 2810 includes a conveyor substrate 2812 such as, but not limited to, a mesh, fibers, filaments, pliable elements or the like. The conveyor substrate 2812 captures and conveys thrombus while at the same time permitting the flow of liquids, such as blood. The thrombus conveyor 2810 including the substrate 2812 is pliable and configured to evert as shown in FIG. 28 (e.g., the everted or deployed configuration of the conveyor 2810). An exterior surface inversion 2842 is provided between the interior surface of 2822 of the conveyor 2810 and the exterior surface 2824 of the conveyor. As discussed herein, the exterior surface inversion 2842 is the transition location of the thrombus conveyor 2810 as the conveyor 2810 everts, for instance to engage with and shuttle thrombus into the thrombus conveyor 2810 and shuttle the thrombus toward the conveyor sheath 2806. A conveyor mouth 2818 of the thrombus conveyor 2810 extends from the exterior surface inversion 2842 toward the conveyor shaft distal portion 2826 (where the conveyor 2810 couples with the portion 2826).

Referring again to FIG. 28, the sheath lumen 2808 of the conveyor sheath 2806 (a component of the conveyor catheter 2802) receives a basket catheter 2836. As further shown in FIG. 28, the conveyor shaft 2804 (also of the conveyor catheter 2802) is received within the sheath lumen 2808 and includes its own conveyor shaft lumen 2834. The basket catheter 2836 is, in this example, provided within the conveyor shaft lumen 2834 of the conveyor shaft 2804. Accordingly, the basket catheter 2836 is nested within the conveyor shaft 2804 and the conveyor sheath 2806. The basket catheter 2836 extends through the thrombus conveyor 2810 coupled between the conveyor sheath 2806 and the conveyor shaft 2804.

As shown in FIG. 28, the basket catheter 2836 includes a basket shaft 2838 positioned within the conveyor shaft lumen 2834 of the conveyor shaft 2804. As shown, the at least one collection basket 2840 is coupled with the basket shaft 2838 and is provided in a deployed configuration distal to the conveyor mouth 2818 of the thrombus conveyor 2810. In other examples a plurality of collection baskets 2840 are coupled with and deployed from the basket shaft 2838. The one or more collection baskets 2840 are constructed with deformable materials, such as shape memory alloys, that are biased toward deployment as shown in FIG. 28, for instance, to positions the baskets 2840 in proximity to vessel walls and to facilitate capture of thrombus. In another example, the basket catheter 2836 includes a pull wire, nested catheter, rod or the like configured to manipulate the collection basket 2840 between the deployed configuration shown and a retracted configuration to facilitate delivery in the conveyor catheter 2802 to a treatment location.

In operation, collection basket 2840 (or baskets) is distally delivered relative to target thrombus in a vessel. For instance, the collection basket 2840 is in a retracted configuration and delivered through the conveyor catheter 2802 or a delivery catheter. The collection basket 2840 is expanded to the deployed configuration, for example because of withdrawal of the delivery catheter or conveyor catheter 2802. The conveyor catheter 2802 is deployed from a delivery catheter proximal to the target thrombus. The collection basket 2840 is proximally withdrawn and guides the thrombus toward to the conveyor mouth 2818.

With the target thrombus captured between the conveyor mouth 2818 and the proximally withdrawn collection basket 2840 (still distal to the thrombus conveyor 2810) the basket catheter 2836 is anchored to the conveyor shaft 2804, for instance with a clamp, fastener or the like. Accordingly, translating movement of the conveyor shaft 2804 (relative to the conveyor sheath 2806) also moves the basket catheter 2836 and its collection basket 2840. For instance, the collection basket 2840 is static relative to the conveyor shaft 2804 and the interior surface of 2822 of the thrombus conveyor 2810 coupled with the conveyor shaft 2804. The thrombus is guided between the basket 2840 and the thrombus conveyor 2810 toward the conveyor mouth 2818.

As shown in FIG. 28, the conveyor mouth 2810 extends from the exterior surface inversion 2842 toward the conveyor shaft distal portion 2826. The inversion 2842 is a fold or joint of the exterior surface 2824 of the conveyor as it transitions from facing away from the conveyor shaft (everted) to facing the conveyor shaft 2804 (inverted), for instance with the conveying motion of the thrombus conveyor 2810. The conveyor mouth 2818 is pliable and compresses the thrombus in a compliant manner as the thrombus moves proximally with the conveyor substrate (e.g., each is translationally static relative to the other). Additionally, the conveyor mouth 2818 has a conveyor mouth profile (e.g., a pocket, recess, cavity, bowl or the like) that readily receives thrombus and tapers from the exterior surface inversion 2842 toward the conveyor shaft anchor 2832.

As described herein, the static interface between the thrombus and the conveyor 2810 minimizes shearing and lineal compression of thrombus that otherwise generates plugging. Proximal movement of the conveyor shaft 2804 and the basket catheter 2836 guides the thrombus further into the thrombus conveyor 2810 and at the same time inverts the everted portion of the thrombus conveyor 2810, the exterior surface 2824. The exterior surface 2824 of the thrombus conveyor 2810 inverts at the exterior surface inversion 2842 as the conveyor shaft 2804 is proximally withdrawn relative to the conveyor sheath 2806. As the collection basket 2840, thrombus, basket shaft 2838 (of the basket catheter 2836) and the conveyor shaft 2804 are proximally withdrawn the thrombus is static relative to at least the interior surface 2822 of the conveyor 2810 facing and engaging the thrombus. At the same time the exterior surface 2824 of the thrombus conveyor 2810 is inverting at the exterior surface inversion 2842 (proximate the conveyor mouth 2818) and transitioning to the interior surface 2822 of the conveyor engaged with the guided thrombus.

Continued proximal movement of the conveyor shaft 2804 relative to the conveyor sheath 2806 further inverts the thrombus conveyor 2810 as the thrombus is guided into the conveyor 2810. The thrombus is static relative to the conveyor 2810 and the collection basket 2840 (coupled to the conveyor shaft 2804) as the conveyor 2810 is inverted around the thrombus. The thrombus is gradually enveloped by the inverting thrombus conveyor 2810 and the collection basket 2840 is optionally enveloped if inversion of the conveyor 2810 continues to thereby enclose the thrombus.

The enclosed thrombus is radially compressed and optionally lengthened as the thrombus conveyor 2810 deflects inwardly and stretches as it approaches the conveyor sheath 2860. At the conveyor sheath 2860 the compressed thrombus is readily delivered into the sheath lumen 2808 (proximate to the conveyor sheath anchor 2830 or sheath mouth) without plugging.

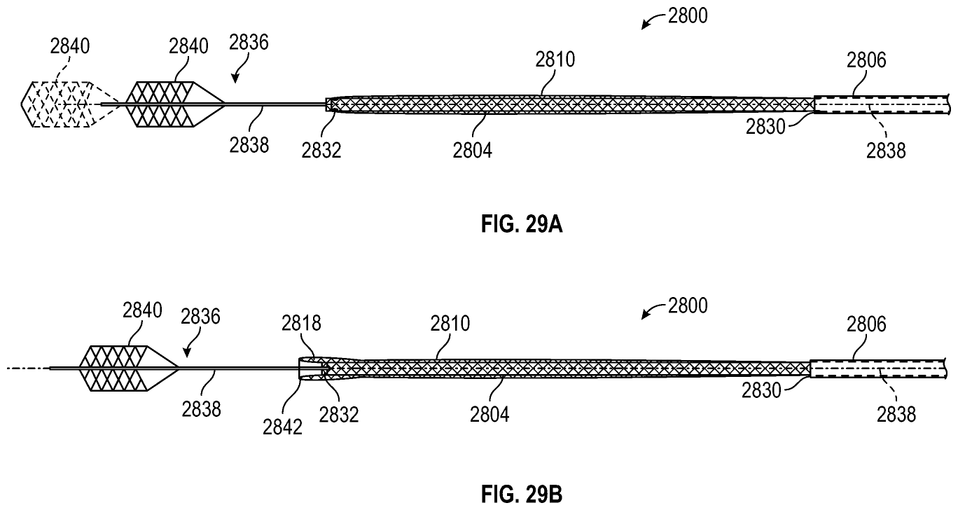

FIGS. 29A and 29B illustrate the thrombus capture and conveyor system 2800 with the thrombus conveyor 2810 in everted configurations. In the example shown in FIG. 29A, the thrombus conveyor is in a fully everted configuration, with the thrombus conveyor everted between each of the conveyor sheath anchor and the conveyor shaft anchor. An example basket catheter 2836 is shown distal to the thrombus conveyor 2810. The basket catheter 2836 includes at least one collection basket 2840. As shown in broken lines the basket catheter 2836 optionally includes multiple collection baskets 2840. As further discussed herein, the distal collection basket 2840 is optionally provided on a separate basket shaft 2838 to facilitate independent movement of the collection baskets 2840 (e.g., for separation of thrombus, and separate enveloping and shuttling with the thrombus convey 2810).

Referring again to FIG. 29A, as previously described the thrombus conveyor 2810 is an everted configuration, in this example a fully everted configuration. For instance, the conveyor shaft 2804 and conveyor sheath 2806 positioned as shown, and delivered through an introducer, delivery catheter or the like. As the conveyor shaft 2804 and sheath 2806 are deployed from the delivery catheter the thrombus conveyor is fully everted between the conveyor sheath anchor 2830 and the conveyor shaft anchor 2832. To begin inversion, the conveyor shaft 2804 is moved proximally relative to the conveyor sheath 2806. The movement forms the exterior surface inversion 2842 (see FIG. 28) as a portion of the thrombus conveyor 2810 proximate to the conveyor shaft anchor 2832 is inverted to the interior surface of the conveyor 2822 (FIG. 28).

FIG. 29B shows the thrombus conveyor 2810 optionally deployed in another everted configuration, for instance with a portion of the conveyor 2810 inverted at the exterior surface inversion 2842. In this example, the thrombus conveyor 2810 is delivered through an introducer or delivery catheter to a treatment location with the conveyor 2810 in the configuration shown (e.g., everted with a partial inversion). For instance, the conveyor shaft 2804 is proximally retracted relative to the conveyor sheath 2806 to initiate the exterior surface inversion 2842. The conveyor shaft 2804 is then optionally clamped to the conveyor sheath 2806 to maintain the thrombus conveyor 2810 in this everted configuration during delivery (e.g., through the vasculature, delivery catheter or the like). With the thrombus conveyor 2810 everted as shown a conveyor mouth 2818 is provided that extends from the exterior surface inversion 2842 toward the conveyor shaft anchor 2832.

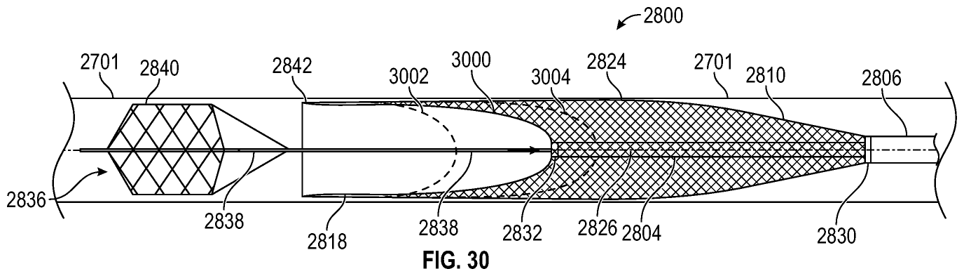

FIG. 30 is a detailed view of the thrombus capture and conveyor system 2800 showing the conveyor mouth 2819 with various conveyor mouth profiles 3000 (solid lines), 3002, 3004 (dashed lines). The conveyor mouth 2818 extends from the exterior surface inversion 2842 toward the conveyor shaft distal portion 2826. The inversion 2842 is a fold or joint of the thrombus conveyor 2810 as it transitions from facing away from the conveyor shaft 2804 (everted) to facing the conveyor shaft 2804 (inverted). As discussed herein, proximal movement of the conveyor shaft 2804 relative to the conveyor sheath 2806 transitions the thrombus conveyor 2810 between the everted and inverted configurations and at the same time shuttles thrombus (and a captured collection basket) toward the conveyor sheath 2806.

In one example, with at least portions of the thrombus conveyor 2810 inverted and everted the conveyor exterior surface 2824 is directed away from the conveyor shaft. A bias is optionally applied to the exterior surface 2824 to bias it outwardly and into conformance with the vessel 2701 through one or more of the conveyor substrate materials, construction of the conveyor (e.g., braid pattern) or the inversion of the conveyor (e.g., transition between the everted and inverted configurations). The biased thrombus conveyor 2810 pliably conforms to the contours of the vessel 2710 and thereby provides a similarly conforming exterior surface inversion 2842 and (a proximate portion of the) conveyor mouth 2818 extending from the inversion 2842. The conforming shape of the conveyor 2810, inversion 2842 and the conveyor mouth 2818 facilitate the capture of thrombus and minimizes the escape of particulate around the conveyor 2810.

A plurality of example conveyor mouth profiles 3000, 3002, 3004 are shown in FIG. 30. The conveyor mouth profile includes one or more of shape, size, depth or the like of the thrombus conveyor 2810 configured to receive thrombus. In one example, the mouth profile 3000 is modulated through movement of the conveyor shaft 2804 (e.g., proximal relative to the conveyor sheath). The movement, as shown in FIG. 30, transitions the mouth profile between one or more configurations such as profiles 3000, 3002, 3004 as well as additional profiles with varied movement of the shaft 2804. Example mouth profiles 3000, 3002, 3004 are shown with solid and dashed lines in FIG. 30. For instance, the mouth profiles of the conveyor 2818 are shaped with a clinician specified depth, shape or the like while the collection basket 2840 remains deployed away from the thrombus conveyor 2810 (e.g., while the conveyor shaft 2804 is moved). After setting of the mouth profile to a specified shape the collection basket 2840 is moved proximally toward the thrombus conveyor 2810 with thrombus therebetween. In one example, with a relatively deep mouth profile 3000-3004 the reception of thrombus is facilitated within the thrombus conveyor 2810. Optionally, the collection basket 2840 is fixed at an offset distance (described herein) to minimize axial compression of the captured thrombus, and the thrombus conveyor 2810 is then inverted as discussed herein to convey the thrombus toward the conveyor sheath 2806. The collection basket 2840 moves with the conveyor shaft 2804 based on selective clamping between the basket and the shaft and the basket 2840 continues to guide thrombus into the thrombus conveyor 2810 for shuttling to the conveyor sheath 2806.

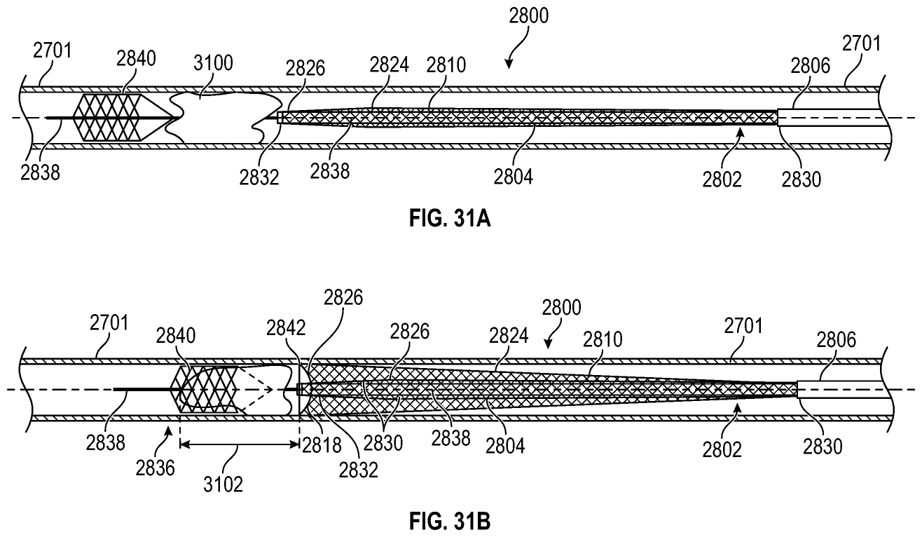

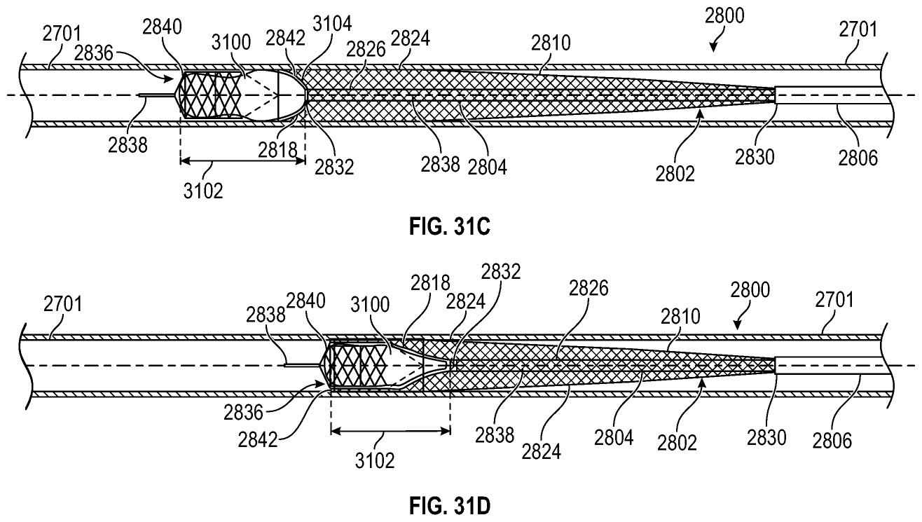

Referring now to FIG. 31A, the thrombus capture and conveyor system 2800 is shown with the thrombus conveyor 2810 initially everted (as shown in FIG. 29A). As shown in FIG. 29B the thrombus conveyor 2810 is then partially inverted with a portion of the exterior surface inverted to form the mouth profile 3000 of the conveyor mouth 2818 for reception of the 3100 therein. For instance, the conveyor catheter 2804 is moved proximally relative to the conveyor sheath 2806 to initiate folding inversion of the thrombus conveyor 2810 at the exterior surface inversion 2842 (see FIG. 31B).

The thrombus 3100 is between the collection basket 2804 and the thrombus conveyor 2810. For example, the collection basket 2840 is delivered within an associated sheath or catheter past the thrombus 3100 and then deployed. As discussed herein, the collection basket 2840 is in other examples deployed past a proximal portion of the thrombus, for instance within the thrombus itself (e.g., like an anchor) or as shown here with the basket deployed distal to the thrombus.

FIG. 31B shows the thrombus conveyor 2810 in an everted configuration, for instance with the conveyor mouth 2818 having a conveyor mouth profile 3104 provided with initiation of a partial inversion at the exterior surface inversion 2842. In one example, the partial inversion biases the conveyor exterior surface 2824 into intimate engagement along the vessel 2701 wall. In another example, the conveyor 2810 deploys by way of shape memory materials, biased movement of the conveyor substrate (e.g., a self-expanding braid) or the like into the deployed configuration. Inversion of the thrombus conveyor 2810 forms the conveyor mouth 2818, and optionally facilitates control of the conveyor mouth profile. As previously described, the conveyor shaft 2804 is moved relative to the conveyor sheath 2806 to control the profile of the conveyor mouth 2818 through inversion of the thrombus conveyor 2810.

In the configuration shown in FIG. 31B, the thrombus 3100 is captured between the collection basket 2840 and the thrombus conveyor 2810. For instance the deployed collection basket 2840 is proximally moved with its basket shaft 2838 toward the thrombus conveyor 2810 including the conveyor mouth 2818. The thrombus 3100 is captured without longitudinal compression (other than incidental compression as the basket 2840 and conveyor 2810 engage with the thrombus 3100) to minimize plugging, for instance when received at the conveyor sheath 2806.

In one example, the collection basket 2840 and the conveyor mouth 2818 of the thrombus conveyor 2810 are arranged at an offset distance 3102 that corresponds to a length of the thrombus 3100 or the length between the conveyor 2810 and a proximal portion of the collection basket 2840, for instance viewed under fluoroscopy. In another example, the offset distance 3102 is set by a clinician based on feedback from the system 2800, for instance based on resistance (e.g., feel, haptic feedback or the like) to additional proximal movement of the basket catheter 2836 as the collection basket 2840 and thrombus conveyor 2810 capture the thrombus 3100 therebetween. The offset distance 3102 is fixed, for instance with a clamp that locks the basket catheter 2836 (e.g., the basket shaft 2838) with the conveyor catheter 2802 (e.g., the conveyor shaft 2804). Once locked, movement of either of the basket catheter 2836 or the conveyor catheter 2802 moves the collection basket 2840, thrombus 3100, and the inverted portion (e.g., interior) of the thrombus conveyor 2810 collectively, for instance, together without relative movement therebetween. The offset distance 3102 is maintained, and accordingly proximal movement of the collection basket 2840 guides the thrombus 3100 into the thrombus conveyor for shuttling by the conveyor 2810 as described herein.

FIG. 31C the thrombus conveyor 2810 is further inverted to shuttle the thrombus 3100 and the collection basket 2840 of the basket catheter fixed to the conveyor shaft 2804 of the conveyor catheter 2802 toward the conveyor sheath 2806. For instance the mouth profile of the conveyor mouth 2818 is enlarged (e.g., lengthened) to receipt the thrombus 3100 therein. As the thrombus 3100 is received along the inverted portion of the thrombus conveyor 2810 it engages with the conveyor 2810. With continued inversion of the conveyor 2810 the conveyor and the thrombus 3100 move together without relative movement therebetween. Instead, the thrombus conveyor 2810 moves the thrombus 3100 toward the conveyor sheath 2806 in the manner of a shuttle (conveyor belt or the like) transporting the thrombus 3100 toward the sheath.

As shown in FIG. 31C, the everted exterior surface 2824 remains engaged with the vessel 2701 wall even while the conveyor 2810 is inverted. The exterior surface inversion 2842 moves proximally with proximal movement of the conveyor shaft 2804, and the exterior surface 2824 folds or rolls inwardly (inverts to face toward the conveyor shaft 2804) while the remaining everted exterior surface 2824 is directed away from the conveyor shaft 2804 and toward the vessel 2701 wall.

With gradual inversion of the thrombus conveyor 2810 the thrombus 3100 remains captured between the collection basket 2840 and the conveyor 2810. In this example, the offset distance 3102 is maintained (e.g., with the conveyor shaft 2804 anchored to the basket shaft 2838), and accordingly the thrombus 3100 is maintained between the collection basket 2840 and the conveyor 2810 without axial compression therebetween. Instead, the thrombus 3100 is guided and engaged with the conveyor 2810 as the conveyor is inverted and conveyed toward the conveyor sheath 2806. As shown in FIG. 31C the thrombus 3100 is enveloped by the proximally moving inverted portion of the thrombus conveyor 2810 (e.g., the interior) and the thrombus 3100 and the thrombus conveyor 2810 move together toward the sheath 2806.

Referring now to FIG. 31D, the exterior surface 2824 of the thrombus conveyor 2810 continues to transition from facing outwardly to facing inwardly. As shown, the exterior surface 2824 continues to invert (e.g., roll, fold, peel or the like) at the exterior surface inversion 2842 to transition from facing away from the conveyor shaft 2804 to facing toward the conveyor shaft 2804. As the thrombus conveyor transitions from exterior to interior facing, the exterior surface 2824 peels away from the vessel 2701 wall.

The inverted thrombus conveyor 2810 surrounds the thrombus 3100 with inversion and conveys the thrombus 3100 proximally. The conveyor 2810 provides passive (radial) compression of the thrombus 3100 as the conveyor 2810 moves toward the conveyor sheath 2806 and tapers toward the opening of the conveyor sheath 2806. Because longitudinal compression is minimized (the thrombus may actually lengthen) the formation of thrombus plugs is minimized. Continued inversion of the thrombus conveyor 2810 conveys the captured thrombus 3100 toward the conveyor sheath 2806. The thrombus 3100 travels collectively with the thrombus conveyor 2810 (e.g., thrombus 3100 and conveyor 2810 are relatively static), and accordingly the thrombus experiences minimal shearing and longitudinal compression that may otherwise cause compression of the thrombus, snagging with the conveyor substrate, shearing of particulate from the thrombus 3100 or the like. Instead, the thrombus conveyor 2810 shuttles the captured thrombus toward the conveyor sheath 2806.

As further shown in FIG. 31D, in this example, the offset distance 3102 is maintained. The collection basket 2840 moves inside the thrombus conveyor 2810, is enveloped by the inverting conveyor 2810 and moves with the conveyor shaft 2804, thrombus 3100 and the inverted portion of the conveyor 2810 toward the conveyor sheath 2806.

At the distal end of the conveyor sheath 2806 the thrombus conveyor 2810 and the thrombus 3100 are slidably received within the sheath lumen 2808 (see FIG. 28). The conveyor catheter 2802 with the thrombus 3100 within the conveyor sheath 2806 is withdrawn through the vasculature to retract the thrombus from the treatment location. Optionally, aspiration pulls the thrombus 3100 from the thrombus conveyor 2810. In another example, the thrombus conveyor 2810 is withdrawn from a delivery catheter, cleaned of the thrombus 3100, and the conveyor 2810 is reset for additional thrombus removal. For example, the conveyor shaft 2804 is distally moved relative to the conveyor sheath 2806 to re-evert the thrombus conveyor 2810. The re-everted conveyor 2810 is then navigated to the treatment location (including a different treatment location) and the procedure described herein is repeated.

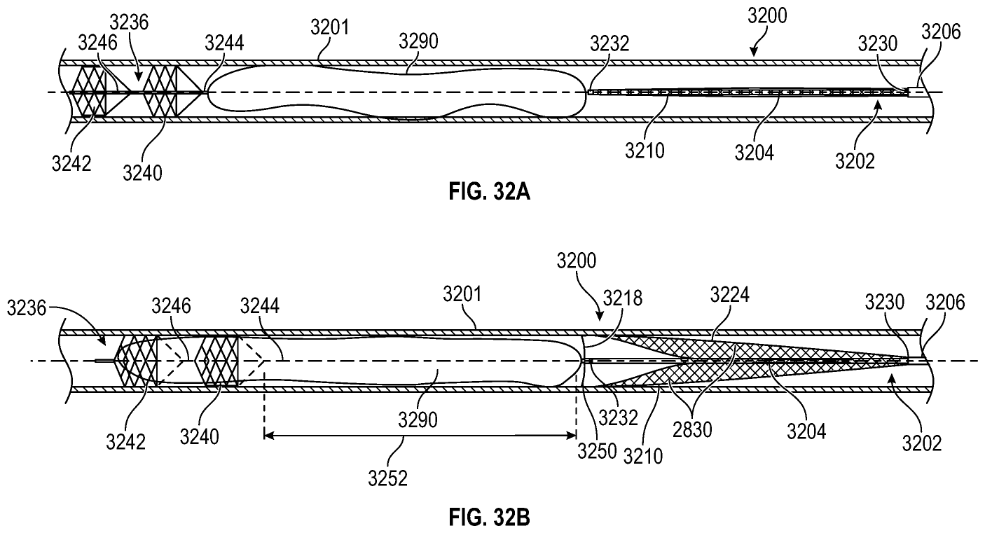

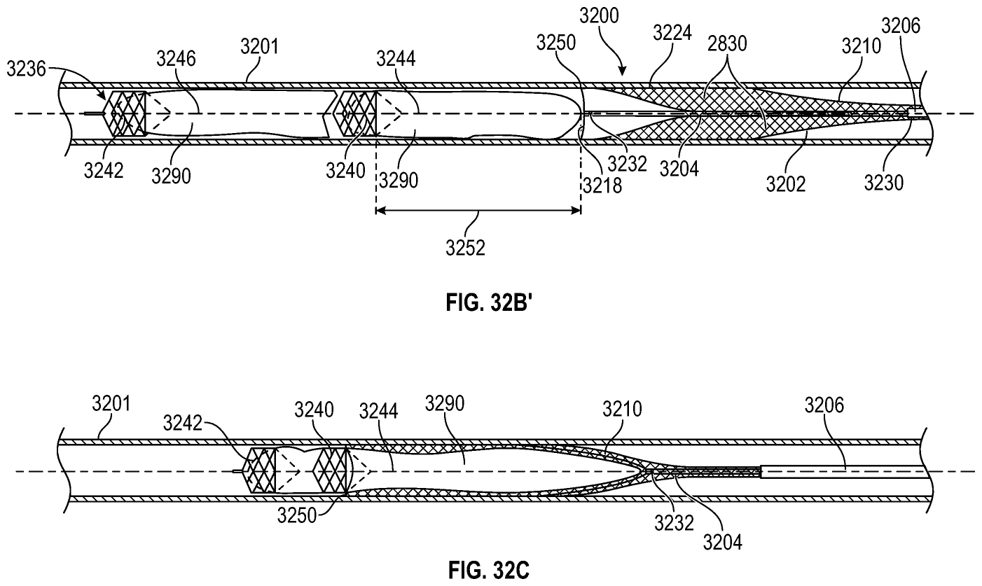

FIGS. 32A-32C show another example of a thrombus capture and conveyor system 3200. In this example the system 3200 shown in FIG. 32A includes a basket catheter 3236 having two or more collection baskets. As shown, the example catheter 3236 includes first and second collection baskets 3240, 3242, with the collection basket 3240 proximal relative to the basket 3242. The collection baskets 3240, 3242 are coupled along a basket shaft. In one example the basket shaft is a unitary shaft 3244 and coupled with each of the baskets 3240, 3242. This permutation is shown in FIG. 32B and permits the deployment of one or more collection baskets 3240 within the thrombus to anchor the basket catheter 3236 in the thrombus 3290 and facilitate guidance of the thrombus toward the thrombus conveyor 3210. In another example, the collection basket 3242 (the distal basket) is coupled with a component basket shaft 3246 nested within a component basket shaft 3244 having the collection basket 3240. Accordingly, the collection baskets 3240, 3242 and their associated shafts 3244, 3246 are movable relative to each other. As described herein, relative movement permits the separation (or partitioning) of the thrombus 3290 into smaller component thrombus for withdrawal from the vessel 3201.

Referring again to FIG. 32A, the thrombus 3290 is initially between the collection baskets 3240, 3242 and the thrombus conveyor 3210. For instance, the collection baskets 3240, 3242 are delivered within an associated sheath or catheter past the thrombus 3290 and then deployed. One or more of the collection baskets 3240, 3242 is deployed past a proximal portion of the thrombus and proximal to a distal portion of the thrombus, for instance within the thrombus 3290 itself (e.g., like an anchor), as shown in FIG. 32B below.

The thrombus conveyor 3210 is initially everted, and as shown in FIG. 32B partially inverted with a portion of the exterior surface inverted to form the mouth profile of the conveyor mouth 3218 for reception of the thrombus 3290 therein. For instance, the conveyor shaft 3204 is moved proximally relative to the conveyor sheath 3206 to deflect the thrombus conveyor 3210 and initiate inversion of the conveyor 3210 at the exterior surface inversion 3250 (see FIG. 32B). In one example, the inversion biases the conveyor exterior surface into intimate engagement along the vessel wall and minimizes the passage of thrombus past the conveyor 3210 while at the same time permitting fluid flow through the conveyor substrate (e.g., a mesh, filaments, braid or the like).

Referring now to FIG. 32B the first collection basket 3240 is deployed within the thrombus 3290. For instance, a sheath is withdrawn from the basket 3240 while positioned within the thrombus 3290. In one example, the collection basket 3240 provides an engagement feature, such as a cleat, barb, anchor or the like (including the basket itself) that engages with the thrombus 3290 and provides a robust coupling to facilitate guidance toward the thrombus conveyor 3210. In another example, the proximal collection basket 3240 separates the thrombus 3290 into component portions (see FIG. 32B′) to facilitate the conveyance of the component portions by the conveyor 3210.

Referring again to FIG. 32B, the second (distal) collection basket 3242 is deployed distally relative to the thrombus 3290. The second collection basket 3242 works in cooperation with the first collection basket 3240 to guide the thrombus 3290 toward the thrombus conveyor 3210. In an example like that shown in FIG. 32B′ the first collection basket 3240 separates the thrombus 3290 into component portions, and the second collection basket 3242 retains the second (distal) portion of the thrombus 3290 while the first (proximal) portion is conveyed with the conveyor 3210. The second collection basket 3242 then guides the second thrombus portion into the conveyor 3210.

FIG. 32B illustrates an example offset distance 3252. The offset distance 3252 corresponds to a length of the thrombus 3290 or the length between the conveyor 3210 and a proximal portion of the collection basket (the proximal collection basket 3240 in this example). Optionally, the system 3200 and thrombus 3290 are viewed under fluoroscopy to determine the offset distance 3252. In another example, the offset distance 3252 is set by a clinician based on feedback from the system, for instance based on resistance (e.g., feel, haptic feedback or the like). The offset distance 3252 is maintained between the thrombus conveyor 3210 and the collection basket 3240 (or baskets 3240, 3246), for instance with a clamp that locks the basket catheter 3236 (e.g., the collection shaft) with the conveyor catheter 3202 (e.g., the conveyor shaft). Accordingly, the basket 3240 (and option basket 3242) and the conveyor 3210 move collectively to guide the thrombus 3290 to the conveyor 3210 and initiate shuttling of the thrombus 3290 with the conveyor 3210.

As shown in FIG. 32C, the baskets 3240, 3242 cooperate with the thrombus conveyor 3210 to guide the thrombus 3290 toward the conveyor sheath 3206. The baskets 3240, 3242, coupled with the basket shaft 3244 move with the thrombus conveyor 3210 as the conveyor inverts from the everted configuration. As previously discussed, the baskets are fixed relative to the basket shaft 3244 to ensure the thrombus 3290 is guided toward the sheath 3206 while minimizing relative movement between the thrombus 3290 and the conveyor 3210 (that may cause shearing, plugging or the like). Instead, the thrombus 3290 is shuttled along with the moving thrombus conveyor 3210 toward the conveyor sheath 3206.

The thrombus conveyor 3210 inverts at the exterior surface inversion 3250 with the exterior surface of the conveyor 3210 transitioning from facing outwardly to facing inwardly. The exterior surface of the thrombus conveyor 3210 peels away from the vessel 3201 wall with inversion while the portion of the everted exterior surface proximal to the exterior surface inversion 3250 is coupled along the vessel wall. The exterior surface continues to invert (e.g., roll, fold, peel away from the vessel 3201 wall or the like) and transitions from facing away from the conveyor shaft 3204 to facing toward the conveyor shaft 3204. Continued inversion of the thrombus conveyor 3210 conveys the captured thrombus 3290 toward the conveyor sheath 3206.

The thrombus 3290 travels collectively with the thrombus conveyor 3210, and accordingly experiences minimal shearing that may otherwise cause compression of the thrombus, plugging, snagging with the conveyor substrate or the like. Instead, the inverted thrombus conveyor 3210 surrounds the thrombus 3290 and shuttles the thrombus proximally toward the conveyor sheath 3206. The conveyor provides passive compression of the thrombus as the conveyor moves toward the conveyor sheath. Because longitudinal compression is minimized (the thrombus may actually lengthen) the formation of the dense plugs is minimized.

Optionally, the offset distance 3252 (see FIG. 32B) is maintained. The collection baskets 3240, 3242 move inside the remaining everted portion of the thrombus conveyor 3210 while maintaining the distance 3252 between the basket 3240 and the conveyor shaft 3204 (e.g., the conveyor shaft anchor 3232 proximate to a distal portion of the conveyor shaft 3204). Accordingly, the thrombus 3290 is maintained between the baskets 3240, 3242 and the conveyor shaft 3204 without relative movement to the conveyor 3210. Instead, the thrombus 3290 is passively compressed and delivered toward the conveyor sheath 3206 along with the collection baskets 3240, 3242, the conveyor shaft 3204 and the inverted (interior facing) portion of the conveyor 3210.

In an example shown in FIG. 32B′, with the first collection basket 3240 movable relative to the second collection basket 3242, a first clamp locks the first collection basket 3240 (and its basket shaft 3244) to the thrombus conveyor 3210. The second collection basket 3242 and its basket shaft 3246 remain decoupled (and movable) relative to the conveyor 3210 and the first basket 3240. In this example, the second collection basket 3242 remains deployed with the partitioned thrombus portion captured with the basket 3242 to retain a second portion of the thrombus 3290 while the remainder of the system 3200 moves collectively relative to the second collection basket (and its associated basket shaft) to convey the first thrombus portion.

In one example, the basket shaft 3244 is fixed (e.g., clamped, locked or the like) with the thrombus conveyor 3210, for instance with the conveyor shaft 3204. As the conveyor shaft 3204 or the basket shaft 3244 is moved, for instance proximally, the other of the basket shaft 3244 (and its basket 3240) and the conveyor shaft 3204 moves in correspondence. As shown in FIG. 32B′, the offset distance 3252 is maintained between the basket 3240 and the conveyor mouth 3218 to ensure the thrombus 3290 (portion with the first collection basket 3240) is guided into the conveyor and shuttled by the conveyor 3210 toward the conveyor sheath 3206. The thrombus conveyor 3210 continues to invert (e.g., roll, fold, peel from the vessel wall or the like) and accordingly shuttles the thrombus 3290 into and through the conveyor sheath 3206. The second portion of the thrombus 3290 is retained with the collection basket 3242. By partitioning the thrombus 3290 the system 3200 is able to convey and deliver manageable portions of the thrombus 3290, for instance, with smaller thrombus conveyors 3210, baskets, conveyor catheter components (e.g., sheath 3206, shaft 3204) or the like.

Because the thrombus conveyor 3210 is coupled at its conveyor distal end with the conveyor shaft 3204 and at its conveyor proximal end is coupled with the conveyor sheath 3206 the conveyor 3210 is readily re-deployed (e.g. everted) to the configuration shown in FIG. 32B′ after having withdrawn the first basket 3240 with the first portion of the thrombus 3290. Re-deployment is conducted in an example without removing the conveyor 3210 from the vessel to manually evert the conveyor 3210. The second collection basket 3242 and remaining second portion of the thrombus 3290 are then guided toward the conveyor mouth 3218 in a similar manner to the first collection casket 3240 (e.g., with proximal movement of the basket shaft 3246). Optionally, once an offset distance 3252 is reached, the basket shaft 3246 is fixed to the conveyor shaft 3204, and the second collection basket 3242, the second portion of the thrombus 3290, and the thrombus conveyor 3210 move together to shuttle the thrombus into and through the conveyor 3210 as it inverts.

In another example, the thrombus capture and conveyor system 3200 includes the first collection basket 3240 without the second collection basket 3242. The first collection basket 3240 is deployed as shown in FIG. 32B′ to separate the thrombus 3290 and convey the first thrombus portion into and through the thrombus conveyor 3210, and the conveyor 3210 shuttles the thrombus into the conveyor sheath 3206. The basket shaft 3244 and the first basket 3240 are optionally withdrawn from the system 3200, for instance through the conveyor sheath 3206. In one example, the basket 3240 is cleaned (e.g., aspirated) to remove thrombus and is navigated in a delivery catheter, the conveyor sheath 3206 or the like to the treatment site for continued operation in a thrombectomy procedure. For instance, after removal of thrombus from the first basket 3240 (or use of a different basket) the basket 3240 is re-deployed distal to the second thrombus portion 3290, and the second portion is guided to the reset thrombus conveyor 3210 for shuttling of the second portion into the conveyor sheath 3206.

As noted herein above, because the thrombus conveyor 3210 is coupled with the conveyor shaft 3204 and the conveyor sheath 3206 the conveyor 3210 is readily re-deployed (e.g. everted) to the configuration shown in FIG. 32B′ after withdrawal of the first basket 3240 with the first portion of the thrombus 3290. With the thrombus conveyor 3210 re-deployed and the collection basket 3240 positioned distal to the remaining (second) portion of thrombus 3290, the capture and shuttling of the thrombus is repeated with inversion of the thrombus conveyor 3210.

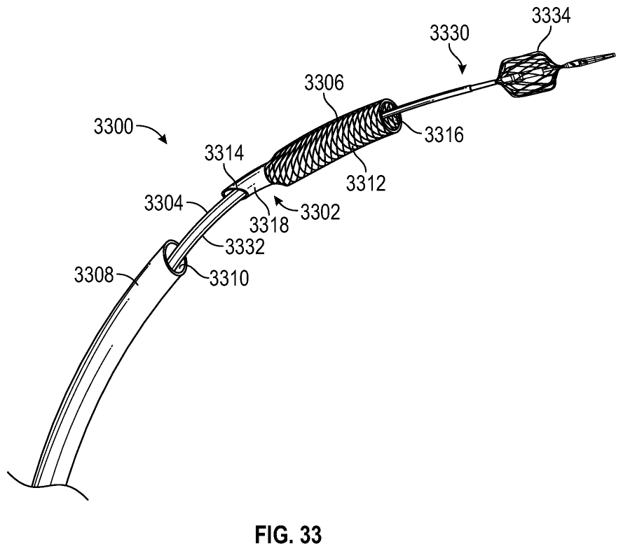

FIG. 33 illustrates another example of a thrombectomy system 3300. The system 3300 includes a trumpet catheter 3302 having a trumpet shaft 3304 coupled with a trump 3306. As shown in FIG. 33, the trumpet shaft 3304 in this example is a stylet, rail, guidewire or the like and has a minimized profile relative to a tubular catheter, sheath or the like. As discussed herein, the trumpet shaft 3304 (and the basket shaft of the basket catheter 3330) assume a minimal proportion of the sheath lumen 3310 of the trumpet sheath 3308 and thereby enhance the available remaining volume of the sheath lumen 3310 for additional catheters, instruments, passage of thrombus or the like.

Referring again to FIG. 33, the trumpet 3306 is constructed with a trumpet substrate 3312. The trumpet substrate 3312 captures thrombus, for instance delivered with the basket catheter 3330, while permitting fluid flow (e.g., blood or the like). The trumpet substrate 3312 includes, but is not limited to, fibers, filaments, elements or the like that are braided, meshed, knitted, non-woven or the like. As further shown in FIG. 33, the trumpet catheter 3302 includes a trumpet shaft anchor 3318 that couples the trumpet 3306 with the trumpet shaft 3304 (e.g., a guide wire, catheter shaft or the like). In this example, the trumpet shaft anchor 3318 includes a catheter port 3314 configured to receive catheters therein. The catheter port 3314 is proximate to a proximal end of the trumpet 3306 and receives catheters and other instruments to facilitate delivery of the catheters and instruments thereon through the trumpet mouth 3316. The catheter port 3314 and the trumpet shaft 3304 facilitate the decreased profile of the trumpet catheter 3302 (e.g., in contrast to a cylindrical catheter coupled with the trumpet 3306) and accordingly enhance available space within the sheath lumen 3310 of the trumpet sheath 3308. As shown in FIG. 33 the profile of the trumpet shaft 3304 and the adjacent basket shaft 3332 are readily received within the sheath lumen 3310 of the trumpet sheath 3308. The trumpet shaft 3304 and the basket shaft 3332 assume a minimal proportion of the sheath lumen 3310 and thereby enhance the remaining proportion of the sheath lumen 3310 for additional catheters, instruments, passage of thrombus or the like.

An example basket catheter 3330 is shown in FIG. 33. The basket catheter 3330 is similar in some regards to the basket catheters previously discussed herein. The basket catheter 3330 includes a basket shaft 3332 coupled with a collection basket 3334. The collection basket 3334 is delivered on the basket shaft 3332 through the trumpet 3306 to a treatment site. For instance, the basket shaft 3332 and the trumpet 3306 are delivered through the catheter port 3314 and through the trumpet mouth 3316 for deployment distal to thrombus with the trumpet 3306 proximal to thrombus (e.g., thrombus is between the trumpet 3306 and the collection basket 3334. In another example, the basket catheter 3330 and the trumpet catheter 3302 are loaded into a delivery catheter or the like together. For instance, the collection basket 3334 is pre-positioned within the trumpet 3306 and the basket 3334 and trumpet 3306 are loaded into the delivery catheter, and advanced through the delivery catheter to a treatment site. Delivery of the collection basket 3334 through a tubular trumpet catheter within the sheath lumen 3310 is thereby unnecessary. Accordingly, the sheath lumen 3310 includes a relatively large volume of available space for the delivery and movement of catheters, instrument, aspiration, infusion, withdrawal of thrombus or the like.

### Various Notes & Examples

Example 1 is an intravascular filter system, comprising: a positioning guidewire; a flexible sleeve positioning tube having a proximal end and a distal end; a flexible capture sleeve attached to said distal end of said flexible sleeve positioning tube, said flexible capture sleeve comprises a woven mesh having an expanded conformation and an unexpanded conformation; a filter catheter having a proximal end and a distal end, said filter catheter defining a central lumen for slidably receiving the positioning guidewire; and a filter assembly having at least one expandable filter attached to said filter catheter at the distal end of the filter catheter, wherein said filter catheter is slidable over said positioning guidewire to pull said filter into the flexible sleeve.

In Example 2, the subject matter of Example 1 optionally includes wherein said filter assembly further comprises: a proximal expandable filter having an expanded conformation and an expanded conformation; a distal expandable filter having an expanded conformation and an expanded conformation; and an intermediate collar positioned between and connection said proximal filter to said distal filter, wherein said intermediate collar is slidably attached to said filter catheter.

In Example 3, the subject matter of any one or more of Examples 1-3 optionally includes wherein said intermediate collar comprises a fenestrated structure having an axially elongated conformation and an axially compressed conformation.

In Example 4, the subject matter of any one or more of Examples 1-3 optionally includes wherein the filter assembly further comprises: a fixed collar fixedly attached to said filter catheter, said fixed collar is attached to one of said proximal filter and said distal filter; and a slidable collar slidably attached to said filter catheter, said slidable collar is attached to the other of said proximal filter and said distal filter.

In Example 5, the subject matter of any one or more of Examples 1˜4 optionally includes wherein the fixed collar and slidable collar comprise fenestrated structure.

In Example 6, the subject matter of any one or more of Examples 1-5 optionally includes a filter delivery catheter defining a central lumen for slidably receiving said filter catheter and said filter assembly; wherein said central lumen of said filter delivery catheter is sized to compress said expandable filter to said unexpanded conformation when said filter assembly is received within said central lumen of said filter delivery catheter.

In Example 7, the subject matter of any one or more of Examples 1-6 optionally includes wherein said filter catheter further comprises: a dilator tip attached to said distal end of said filter catheter, said dilator tip having a tapered tip and a shoulder; wherein said dilator tip is sized such that said shoulder engages said filter delivery catheter when said filter catheter is received within said central lumen of said filter delivery catheter.

Example 8, the subject matter of any one or more of Examples 1-7 optionally includes an intravascular filter system, comprising: a positioning guidewire; an aspiration sheath having a sheath body defining a central lumen and a side path defining a secondary lumen operably connected to said central lumen, said sheath body having a proximal end and a distal end; a filter catheter having a proximal end and a distal end, said filter catheter defining a central lumen for slidably receiving the positioning guidewire; and a filter assembly having at least one expandable filter attached to said filter catheter at the distal end of the filter catheter, wherein said filter catheter is slidable over said positioning guidewire to pull said filter to said distal end of sheath body.

In Example 9, the subject matter of any one or more of Examples 1-8 optionally includes wherein said aspiration sheath further comprises: a hub attached to said proximal end of said sheath body, wherein said hub comprises a hemostasis valve for operably sealing central lumen of said sheath body.

In Example 10, the subject matter of any one or more of Examples 1-9 optionally includes wherein said side path further comprises: a connector for connecting a syringe to said side path to pull a vacuum through secondary lumen of side path and said central lumen of said sheath body; and a valve operable control flow through said side path.

In Example 11, the subject matter of any one or more of Examples 1-10 optionally includes wherein said filter assembly further comprises: a proximal expandable filter having an expanded conformation and an expanded conformation; a distal expandable filter having an expanded conformation and an expanded conformation; and an intermediate collar positioned between and connection said proximal filter to said distal filter, wherein said intermediate collar is slidably attached to said filter catheter.

In Example 12, the subject matter of any one or more of Examples 1-11 optionally includes wherein said intermediate collar comprises a fenestrated structure having an axially elongated conformation and an axially compressed conformation.

In Example 13, the subject matter of any one or more of Examples 1-12 optionally includes wherein the filter assembly further comprises: a fixed collar fixedly attached to said filter catheter, said fixed collar is attached to one of said proximal filter and said distal filter; and a slidable collar slidably attached to said filter catheter, said slidable collar is attached to the other of said proximal filter and said distal filter.

In Example 14, the subject matter of any one or more of Examples 1-13 optionally includes wherein the fixed collar and slidable collar comprise fenestrated structure.

In Example 15, the subject matter of any one or more of Examples 1-14 optionally includes a filter delivery catheter defining a central lumen for slidably receiving said filter catheter and said filter assembly; wherein said central lumen of said filter delivery catheter is sized to compress said expandable filter to said unexpanded conformation when said filter assembly is received within said central lumen of said filter delivery catheter.

In Example 16, the subject matter of any one or more of Examples 1-15 optionally includes wherein said filter catheter further comprises: a dilator tip attached to said distal end of said filter catheter, said dilator tip having a tapered tip and a shoulder; wherein said dilator tip is sized such that said shoulder engages said filter delivery catheter when said filter catheter is received within said central lumen of said filter delivery catheter.

In Example 17, the subject matter of any one or more of Examples 1-16 optionally includes a method for removing a clot, comprising: passing a distal end of a positioning guidewire across a clot located in a blood vessel; sliding a filter catheter over a positioning guidewire such that a filter assembly positioned on a distal end of said filter catheter is located distal to said clot; expanding said filter assembly within said blood vessel distal to said clot; sliding a flexible sleeve catheter over said filter catheter to position a flexible sleeve attached to a distal end of said flexible sleeve; and pulling said expanded filter assembly to draw filter assembly through said clot and into said flexible sleeve.

In Example 18, the subject matter of any one or more of Examples 1-17 optionally includes sliding a filter delivery catheter over said filter catheter and said filter assembly to constrain and collapse said filter assembly for movement past said clot; wherein said filter assembly comprises a fenestrated structure configured to self-expand when unconstrained.

In Example 19, the subject matter of any one or more of Examples 1-18 optionally includes sliding said filter catheter such that a dilator tip attached to said distal end of said filter catheter engages a distal end of said filter delivery catheter.

In Example 20, the subject matter of any one or more of Examples 1-19 optionally includes sliding a flexible sleeve sheath over said flexible sleeve catheter to constrain and collapse said flexible sleeve; wherein said flexible sleeve comprises a woven mesh configured to self-expand when unconstrained.

In Example 21, the subject matter of any one or more of Examples 1-20 optionally includes a thrombus capture and conveyor system comprising: a conveyor sheath extending from a sheath proximal portion to a sheath distal portion; a conveyor catheter slidably received within the conveyor sheath, the conveyor catheter includes: a conveyor shaft extending from a conveyor shaft proximal portion to a conveyor shaft distal portion; and a thrombus conveyor coupled with the conveyor shaft, the thrombus conveyor includes: a conveyor substrate extending annularly from a conveyor proximal end to a conveyor distal end, the conveyor substrate includes interior and exterior surfaces; and the conveyor distal end is coupled with the conveyor shaft distal portion of the conveyor shaft, and the conveyor proximal end is coupled with the sheath distal portion of the conveyor sheath; and wherein the thrombus conveyor is configured to reversibly transition between everted and inverted configurations with movement of the conveyor catheter relative to the conveyor sheath: in the everted configuration the exterior surface of the thrombus conveyor is directed away from the conveyor shaft; and in the inverted configuration the exterior surface of the thrombus conveyor is directed toward the conveyor shaft.

In Example 22, the subject matter of any one or more of Examples 1-21 optionally includes wherein the thrombus conveyor is configured to reversibly transition: from the everted configuration to the inverted configuration with proximal movement of the conveyor catheter relative to the conveyor sheath; and from the inverted configuration to the everted configuration with distal movement of the conveyor relative to the conveyor sheath.

In Example 23, the subject matter of any one or more of Examples 1-22 optionally includes wherein the thrombus conveyor includes a pliable conveyor mouth between the conveyor proximal and distal ends.

In Example 24, the subject matter of any one or more of Examples 1-23 optionally includes wherein the thrombus conveyor is configured to passively compress thrombus within the thrombus conveyor and between the pliable conveyor mouth and the conveyor proximal end.

In Example 25, the subject matter of any one or more of Examples 1-24 optionally includes wherein the pliable conveyor mouth extends from an exterior surface inversion of the conveyor substrate toward a conveyor shaft anchor coupling the thrombus conveyor to the conveyor shaft distal portion.

In Example 26, the subject matter of any one or more of Examples 1-25 optionally includes wherein the thrombus conveyor includes an exterior surface inversion between a conveyor sheath anchor and a conveyor shaft anchor, the conveyor sheath anchor couples the conveyor proximal end with the sheath distal portion and the conveyor shaft anchor couples the conveyor distal end with the conveyor shaft distal portion.

In Example 27, the subject matter of any one or more of Examples 1-26 optionally includes a basket catheter received slidably received within thrombus conveyor, the basket catheter includes: a basket shaft extending through the thrombus conveyor; and a collection basket coupled with basket shaft proximate to the conveyor shaft distal portion.

In Example 28, the subject matter of any one or more of Examples 1-27 optionally includes wherein in the everted configuration the collection basket is configured to position captured thrombus between the collection basket and the thrombus conveyor; and as the thrombus conveyor transitions from the everted to the inverted configurations the collection basket and the thrombus conveyor are configured to convey the thrombus toward the conveyor sheath.

In Example 29, the subject matter of any one or more of Examples 1-28 optionally includes wherein the thrombus is static relative to the conveyor substrate engaged with the thrombus as the conveyor transitions between the everted and inverted configurations.

In Example 30, the subject matter of any one or more of Examples 1-29 optionally includes a thrombus capture and conveyor system comprising: a conveyor sheath extending from a sheath proximal portion to a sheath distal portion; a conveyor catheter slidably received within the conveyor sheath, the conveyor catheter includes: a conveyor shaft; a thrombus conveyor coupled between the conveyor shaft and the conveyor sheath, the thrombus conveyor includes: a conveyor substrate extending from a conveyor proximal end to a conveyor distal end; and the conveyor distal end is coupled with the conveyor shaft, and the conveyor proximal end is coupled with the sheath distal portion; a basket catheter movably coupled with the conveyor catheter, the basket catheter includes: a basket shaft extending through the thrombus conveyor; and a collection basket coupled with basket shaft; and wherein the collection basket and the thrombus conveyor include capture and convey configurations: in the capture configuration the collection basket is moved toward the thrombus conveyor with thrombus therebetween; and in the conveyor configuration the collection basket, thrombus and thrombus conveyor move together to convey the thrombus into the conveyor sheath.

In Example 31, the subject matter of any one or more of Examples 1-30 optionally includes wherein the thrombus conveyor is configured to transition between everted and inverted configurations with movement of the conveyor catheter relative to the conveyor sheath; and in the conveyor configuration an exterior surface of the thrombus conveyor transitions from the everted configuration to the inverted configuration, and in the inverted configuration the exterior surface is engaged with the thrombus and moves together with the thrombus into the conveyor sheath.

In Example 32, the subject matter of any one or more of Examples 1-31 optionally includes wherein the exterior surface engaged with the thrombus and the thrombus are static relative to each other and move collectively toward the conveyor sheath in the conveyor configuration.

In Example 33, the subject matter of any one or more of Examples 1-32 optionally includes wherein the collection basket, the thrombus and the exterior surface engaged with the thrombus are static relative to each other and move collectively toward the conveyor sheath.

In Example 34, the subject matter of any one or more of Examples 1-33 optionally includes wherein the thrombus conveyor transitions from the everted to the inverted configuration at an exterior surface inversion.

In Example 35, the subject matter of any one or more of Examples 1-34 optionally includes wherein the thrombus conveyor is configured to passively compress the thrombus between the exterior surface inversion and the conveyor sheath.

In Example 36, the subject matter of any one or more of Examples 1-35 optionally includes wherein the exterior surface inversion is between a conveyor sheath anchor and a conveyor shaft anchor, the conveyor sheath anchor couples the conveyor proximal end with the sheath distal portion and the conveyor shaft anchor couples the conveyor distal end with the conveyor shaft.

In Example 37, the subject matter of any one or more of Examples 1-36 optionally includes a catheter clamp coupled between the conveyor catheter and the basket catheter, and the catheter clamp is configured to lock the basket catheter to the catheter clamp; and in the conveyor configuration the collection basket, thrombus and thrombus conveyor are static relative to each other and move collectively toward the conveyor sheath according to the catheter clamp.

In Example 38, the subject matter of any one or more of Examples 1-37 optionally includes wherein the collection basket includes a first collection basket and a second collection basket distal to the first collection basket.

In Example 39, the subject matter of any one or more of Examples 1-38 optionally includes wherein the basket shaft includes a first basket shaft coupled with the first collection basket and a second basket shaft coupled with the second collection basket, and the second basket shaft is movably nested within the first basket shaft.

In Example 40, the subject matter of any one or more of Examples 1-39 optionally includes a method of capturing and conveying thrombus comprising: navigating at least one collection basket of a basket catheter past a proximal portion of a thrombus; deploying the at least one collection basket; capturing the thrombus between the at least one collection basket and a thrombus conveyor, capturing includes: navigating the thrombus conveyor proximal to the proximal portion of the thrombus, the thrombus conveyor coupled with a conveyor shaft and a conveyor sheath; and moving at least one of the thrombus conveyor or the at least one collection basket toward each other with at least the proximal portion of the thrombus therebetween; and conveying the thrombus into the conveyor sheath with collective movement of the thrombus conveyor and the thrombus, conveying includes: transitioning the thrombus conveyor from an everted configuration to an inverted configuration according to proximal movement of the conveyor shaft coupled with the thrombus conveyor; and collectively moving the at least one collection basket, the thrombus and the inverted thrombus conveyor engaged with the thrombus toward the conveyor sheath.

In Example 41, the subject matter of any one or more of Examples 1-40 optionally includes wherein transitioning the thrombus conveyor and collectively moving includes proximally moving a conveyor shaft relative to the conveyor sheath.

In Example 42, the subject matter of any one or more of Examples 1-41 optionally includes wherein transitioning the thrombus conveyor includes: moving an inverted portion of the thrombus conveyor proximally toward the conveyor sheath; and maintaining an everted portion of the thrombus conveyor static relative to a vessel wall.

In Example 43, the subject matter of any one or more of Examples 1-42 optionally includes wherein collectively moving the at least one collection basket, the thrombus and the inverted thrombus conveyor engaged with the thrombus includes maintaining at least the inverted thrombus conveyor engaged with the thrombus static relative to the thrombus.

In Example 44, the subject matter of any one or more of Examples 1-43 optionally includes wherein collectively moving the at least one collection basket, the thrombus and the inverted thrombus conveyor engaged with the thrombus includes shuttling the thrombus toward the conveyor sheath without relative translational movement between the thrombus and the inverted thrombus conveyor engaged with the thrombus.

In Example 45, the subject matter of any one or more of Examples 1-44 optionally includes wherein navigating the at least one collection basket of the basket catheter past the proximal portion of the thrombus includes navigating the at least one collection basket past a distal portion of the thrombus.

In Example 46, the subject matter of any one or more of Examples 1-45 optionally includes wherein navigating the at least one collection basket of the basket catheter past the proximal portion of the thrombus includes positioning the at least one collection basket within the thrombus; and wherein deploying the at least one collection basket includes deploying the at least one collection basket within the thrombus.

In Example 47, the subject matter of any one or more of Examples 1-46 optionally includes wherein one or more of capturing the thrombus or conveying the thrombus into the conveyor sheath severs the thrombus.

In Example 48, the subject matter of any one or more of Examples 1-47 optionally includes resetting the thrombus conveyor from the inverted configuration to the everted configuration with distal movement of the conveyor shaft relative to the conveyor sheath; redeploying the at least one collection basket distal to remaining thrombus after severing; and repeating capturing and conveying of the remaining thrombus.

In Example 49, the subject matter of any one or more of Examples 1-48 optionally includes wherein the at least one collection basket includes first and second collection baskets, and navigating at least one collection basket includes: navigating the first collection basket past the proximal portion of the thrombus; and navigating the second collection basket past a distal portion of the thrombus.

In Example 50, the subject matter of any one or more of Examples 1-49 optionally includes wherein deploying the at least one collection basket includes: deploying the first collection basket within the thrombus; and deploying the second collection basket distal to the thrombus.

In Example 51, the subject matter of any one or more of Examples 1-50 optionally includes initializing the thrombus conveyor including inverting the thrombus conveyor proximate to a conveyor distal end with proximal movement of the conveyor shaft to form a conveyor mouth.

In Example 52, the subject matter of any one or more of Examples 1-51 optionally includes initializing the thrombus conveyor includes modulating a conveyor funnel profile of the conveyor mouth with proximal movement of the conveyor shaft, the conveyor funnel profile configured to receive one or more of the thrombus or collection basket therein.

In Example 53, the subject matter of any one or more of Examples 1-52 optionally includes wherein initializing the thrombus conveyor includes biasing the thrombus conveyor into engagement with a surrounding vessel.

In Example 54, the subject matter of any one or more of Examples 1-53 optionally includes wherein capturing the thrombus includes locking the at least one collection basket in place relative to the thrombus conveyor by an offset distance to minimize longitudinal compression of the thrombus.

In Example 55, the subject matter of any one or more of Examples 1-54 optionally includes wherein the offset distance is proximate to a length of the captured thrombus, and locking the at least one collection basket in place is after moving at least one of the thrombus conveyor the at least one collection basket toward each other.

In Example 56, the subject matter of any one or more of Examples 1-55 optionally includes wherein collectively moving the at least one collection basket, the thrombus and the inverted thrombus conveyor engaged with the thrombus toward the conveyor sheath includes: shuttling the thrombus with the inverted thrombus conveyor; and plunging the thrombus with the collection basket.

Each of these non-limiting examples can stand on its own, or can be combined in any permutation or combination with any one or more of the other examples.

The above detailed description includes references to the accompanying drawings, which form a part of the detailed description. The drawings show, by way of illustration, specific embodiments in which the present subject matter can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the present subject matter should be determined with reference to the appended claims, along with the full scope of equivalents to which such claims are entitled.

What is claimed is:

1. An intravascular filter system, comprising:
a positioning guidewire;
a flexible sleeve positioning tube having a proximal end and a distal end;
a flexible capture sleeve attached to said distal end of said flexible sleeve positioning tube, said flexible capture sleeve includes fenestration openings, the flexible capture sleeve having an expanded conformation and a contracted conformation;
a filter catheter having a proximal end and a distal end, said filter catheter having a central lumen configured for slidable reception of the positioning guidewire; and
a filter assembly having at least one expandable filter attached to said filter catheter proximate the distal end of the filter catheter, wherein said filter catheter is configured for translation over said positioning guidewire to pull said filter into the flexible capture sleeve.

2. The intravascular filter system of claim 1, wherein said filter assembly further comprises:
a proximal expandable filter having a constrained conformation and an expanded conformation;
a distal expandable filter having a constrained conformation and an expanded conformation; and
an intermediate collar positioned between and connection said proximal filter to said distal filter, wherein said intermediate collar is slidably attached to said filter catheter.

3. The intravascular filter system of claim 2, wherein said intermediate collar comprises a fenestrated structure having an axially elongated conformation and an axially compressed conformation.

4. The intravascular filter system of claim 2, wherein the filter assembly further comprises:
a fixed collar fixedly attached to said filter catheter, said fixed collar is attached to one of said proximal filter and said distal filter; and
a slidable collar slidably attached to said filter catheter, said slidable collar is attached to the other of said proximal filter and said distal filter.

5. The intravascular filter system of claim 2, wherein the fixed collar and slidable collar comprise fenestrated structure.

6. The intravascular filter system of claim 1, further comprising:
a filter delivery catheter defining a central lumen for slidably receiving said filter catheter and said filter assembly;
wherein said central lumen of said filter delivery catheter is sized to compress said expandable filter to said unexpanded conformation when said filter assembly is received within said central lumen of said filter delivery catheter.

7. The intravascular filter system of claim 6, wherein said filter catheter further comprises:
a dilator tip attached to said distal end of said filter catheter, said dilator tip having a tapered tip and a shoulder;
wherein said dilator tip is sized such that said shoulder engages said filter delivery catheter when said filter catheter is received within said central lumen of said filter delivery catheter.

8. An intravascular filter system, comprising:
a positioning guidewire;
an aspiration sheath having a sheath body defining a lumen and a side path defining a side lumen operably connected to said lumen, said sheath body having a proximal sheath end and a distal sheath end;
a filter catheter having a proximal catheter end and a distal catheter end, said filter catheter having a catheter lumen configured for slidable receipt of the positioning guidewire; and
a filter assembly having at least one expandable filter attached to said filter catheter proximate the distal end of the filter catheter, wherein said filter catheter is configured for translation over said positioning guidewire to pull said filter toward said distal end of sheath body.

9. The intravascular filter system of claim 8, wherein said aspiration sheath further comprises:
a hub attached to said proximal end of said sheath body, wherein said hub comprises a hemostasis valve for operably sealing central lumen of said sheath body.

10. The intravascular filter of system of claim 8, wherein said side path further comprises:
a connector for connecting a syringe to said side path to pull a vacuum through secondary lumen of side path and said central lumen of said sheath body; and
a valve operable control flow through said side path.

11. The intravascular filter system of claim 8, wherein said filter assembly further comprises:
a proximal expandable filter having an expanded conformation and an expanded conformation;
a distal expandable filter having an expanded conformation and an expanded conformation; and
an intermediate collar positioned between and connection said proximal filter to said distal filter, wherein said intermediate collar is slidably attached to said filter catheter.

12. The intravascular filter system of claim 11, wherein said intermediate collar comprises a fenestrated structure having an axially elongated conformation and an axially compressed conformation.

13. The intravascular filter system of claim 12, wherein the filter assembly further comprises:
a fixed collar fixedly attached to said filter catheter, said fixed collar is attached to one of said proximal filter and said distal filter; and
a slidable collar slidably attached to said filter catheter, said slidable collar is attached to the other of said proximal filter and said distal filter.

14. The intravascular filter system of claim 12, wherein the fixed collar and slidable collar comprise fenestrated structure.

15. The intravascular filter system of claim 8, further comprising:
a filter delivery catheter defining a central lumen for slidably receiving said filter catheter and said filter assembly;
wherein said central lumen of said filter delivery catheter is sized to compress said expandable filter to said unexpanded conformation when said filter assembly is received within said central lumen of said filter delivery catheter.

16. The intravascular filter system of claim 15, wherein said filter catheter further comprises:
a dilator tip attached to said distal end of said filter catheter, said dilator tip having a tapered tip and a shoulder;
wherein said dilator tip is sized such that said shoulder engages said filter delivery catheter when said filter catheter is received within said central lumen of said filter delivery catheter.

17. A method for removing a clot, comprising:
passing a distal guidewire end of a positioning guidewire across a thrombus located in a blood vessel;
sliding a filter catheter over the positioning guidewire, the filter catheter having filter assembly, and sliding the filter catheter positions a filter assembly distal to the thrombus;
expanding said filter assembly within said blood vessel distal to said clot;
sliding a flexible sleeve catheter over said filter catheter, the flexible sleeve catheter having a flexible sleeve, and sliding the flexible sleeve positions the flexible sleeve proximal to the filter assembly; and
pulling said expanded filter assembly through said thrombus and into said flexible sleeve.

18. The method of claim 17, further comprising:
sliding a filter delivery catheter over said filter catheter and said filter assembly to constrain and collapse said filter assembly for movement past said clot;
wherein said filter assembly comprises a fenestrated structure configured to self-expand when unconstrained.

19. The method of claim 18, further comprising:
sliding said filter catheter such that a dilator tip attached to said distal end of said filter catheter engages a distal end of said filter delivery catheter.

20. The method of claim 17, further comprising:
sliding a flexible sleeve sheath over said flexible sleeve catheter to constrain and collapse said flexible sleeve;
wherein said flexible sleeve comprises a woven mesh configured to self-expand when unconstrained.
